(19) United States
(12) Patent Application Publication
(10) Pub. No.: US 2014/0373851 A1
(43) Pub. Date: Dec. 25, 2014

(54) FILLED INCISE DRAPE

(71) Applicant: 3M INNOVATIVE PROPERTIES COMPANY, St. Paul, MN (US)

(72) Inventors: Nicholas R. Powley, St. Paul, MN (US); Matthew T. Scholz, Woodbury, MN (US); Erin A. Satterwhite, Chatham, NJ (US); Andrew P. Flaherty, Dellwood, MN (US); Ranjani V. Parthasarathy, Woodbury, MN (US)

(21) Appl. No.: 14/369,061

(22) PCT Filed: Dec. 27, 2012
(86) PCT No.: PCT/US2012/071734

§ 371 Date: Jun. 26, 2014

Related U.S. Application Data
(60) Provisional application No. 61/581,308, filed on Dec. 29, 2011.

(51) Int. Cl. A61F 13/40; A61B 19/08

(52) U.S. Cl. CPC A61M 35/006; A61B 19/088; A61B 2019/085
USPC 128/849

(57) ABSTRACT

An article, comprising: an enclosure comprising: a first film layer; a second film layer, wherein the first film layer and the second film layer are sealed together to form at least one interior region for enclosing a liquid; and an adhesive layer disposed on an exterior surface of the first film layer; wherein the at least one interior region is liquid-filled. A method, comprising: providing an enclosure comprising: a first film layer; a second film layer; at least one interior region; and a composite layer comprising the first film layer and an adhesive layer disposed on an exterior surface of the first film layer; wherein: the composite layer is liquid-impervious and flexible; the first film layer and the second film layer are sealed together to form the at least one interior region; the interior region has a sealed outer periphery; and the interior region is liquid-filled; positioning the article over an intended site of operation, with the adhesive layer facing towards the intended site of operation; penetrating each of the first film layer, the at least one interior region, and the composite layer with a surgical device; and penetrating the intended site of operation with the surgical device.

### BACKGROUND

For infection prevention, maintaining sterility is of primary importance in surgical procedures, especially those involving incision. Typically, the procedure for preventing surgical site infection includes showering, washing, shaving, scrubbing, prepping, and draping the surgical site. Each of these tasks is performed to prevent the introduction of pathogenic flora into the surgical site. However, the entire process for maintaining the sterility of the surgical site may be negated by the simple and frequent occurrence of incising through a hair follicle. Hair follicles, no matter how well the skin is washed, scrubbed, and prepped, are rich in bacteria. The act of making the surgical incision (i.e., incising) can release bacteria from many hair follicles and other skin structures. Such amounts of bacteria may be sufficient for causing a surgical site infection.

### SUMMARY

An opportunity exists to create a drape that can aid with controlling and delivering antiseptics at and to a surgical site.

Mastery of controlling and delivering liquids, such as antiseptics at and to the surgical site gives rise to other opportunities. Controlling and delivering lubricants, for example, could be useful to the field of minimally invasive, laparoscopic, or robotic surgery. In such surgeries, a surgical tool is generally percutaneously inserted and advanced and withdrawn repeatedly. Such repetitive motion can increase the risk of infection, damage the adjacent skin and anatomical structures, and result in user fatigue. Thus, an opportunity exists to create an incise drape that can control and deliver lubricants in addition to antiseptics at and to the surgical site. Furthermore, it may be beneficial to deliver other agents to a surgical incision such as: hemostatic agents to prevent or reduce bleeding, anti-inflammatory agents to reduce swelling and post operative pain, chemotactic agents to accelerate capillary ingrowth post surgery, wound healing agents and the like as well as combinations thereof.

Filled incise drapes that can control and deliver therapeutic materials at and to the surgical site are described herein. Exemplary embodiments of filled incise drapes of the present disclosure are generally comprised of layers of liquid-impervious material that substantially encapsulate a therapeutic material there-between (that is to say, a liquid is enclosed within at least one cavity; in some embodiments, the cavity is hermetically sealed). Once applied to the surgical site and appropriately prepared, an incision can be made through the filled incise drape. Upon incising or otherwise puncturing through the filled incised drape, the therapeutic material will be expressed from the drape and delivered into the incision. Release from the drape cavity may be controlled by a variety of factors including the cavity design, the pressure within the cavity, manipulation, or gravity. Thus, this invention enables the delivery of highly effective compositions of therapeutic materials typically not used after the initial incision in traditional surgery, because it would require an additional step, and bacteria from the incision is not typically recognized as a contributor to surgical infections. Filled incision drapes of this disclosure provide a single step for delivering a therapeutic material directly to the surgical incision as it is made.

It is intended that the features of the primary embodiments and primary variations of those embodiments described herein may be permuted and repeated to create other embodiments and variations of similar novelty.

In one primary embodiment, a filled incise drape is comprised of two film layers of material, a first film layer and a second film layer. In typical embodiments, the first film layer is coated with adhesive on an external surface of the filled incise drape, which enables the filled incise drape to adhere to skin at a surgical site. In some embodiments, a release liner layer is disposed on the adhesive layer. The two film layers of material are sealed together around a perimeter to create an interior region (i.e., cavity) between the film layers. The interior region between the two layers is filled with a liquid therapeutic material. Upon incising through this filled incise drape embodiment, the liquid therapeutic material will be delivered into the incision by way of pressure, gravity, the continued act of incising, and any accessing of the surgical site or other external force.

In another primary embodiment, a filled incise drape is comprised of two film layers of material, a first film layer and a second film layer. The first layer is a coated with adhesive on one side, which enables the filled incise drape to adhere to a surgical drape already applied to the surgical site. The two layers of material are sealed together around a perimeter to create an interior region between the first film layer and second film layer. The interior region between the first and second film layers is filled with a therapeutic material. In this embodiment, the first film layer is secured to a patient drape such as a surgical incise drape (e.g., that surgical incise drape available from 3M Co., Maplewood, Minn., under the trade designation “3M IOBAN 2”). Upon incising or otherwise puncturing or cutting through this construct of filled incision drape adhered to the surgical drape, the therapeutic material will be delivered into the incision by way of pressure, gravity, the continued act of incising, and any accessing of the surgical site. This filled incision drape embodiment may vary from the surgical drape in size. For example, while a typical surgical drape must be of sufficient size to broadly cover the area around an incision, the filled incision drape through which the incision will be made may be significantly smaller and more closely representative of the shape and size of the intended incision.

In some embodiments, a percutaneously implanted device such as a Peripherally Inserted Central Catheter (PICC) may be implanted through the filled incise drape, both lubricating and further disinfecting the implanted device. Once the PICC has been fully inserted, a covering (e.g., a dressing) may be applied over the surgical drape of this embodiment and the PICC. The result will then be a drape, PICC, and covering combination still substantially filled with a therapeutic material that will continue to actively prevent infection until the covering is removed. In applications such as this (as well as others), it may be beneficial to also include one or more antiseptics in the adhesive adhered to the skin to further decontaminate the skin surrounding the implanted device.

Any embodiment may be designed to include multiple sealed zones to form a quilt-like appearance or cavity embossed pattern that helps to evenly distribute the therapeutic material to the surgical site. Such sealed zones are important to enable the even distribution of therapeutic material especially when the surgical site is not flat and level, such as a bent knee, a bent elbow, or the back or chest of a patient lying on his side. In some embodiments the individual cavities are hermetically sealed while in other embodiments the cavities may communicate with one or more other cavities via one or more channels.

Any embodiment may be designed to include multiple film layers of material to encapsulate and separate multiple therapeutic materials in separate interior regions. These systems may deliver individual active components or they may deliver ingredients that react or interact with one another. For example, they may deliver components that deliver hydrogen peroxide (e.g. glucose and glucose oxidase from separate compartments) or nitric oxide (such as a nitrosothiol and a pH shift to release NO).

Any embodiment may be designed such that one of the film layers is tensioned when the filled incise drape is applied. Such tension allows the second film layer of material to withdraw from the incision site as the incision is being made. Such functionality would provide clearer access to the incision site. Excessive tensioning that could result in skin blisters should be avoided.

Any embodiment may be designed such that one of the layers is a composite of multiple layers that can be formed to create structures that helps to evenly distribute the therapeutic material to the surgical site. In some embodiments these structures are important to enable the even distribution of therapeutic material, especially when the surgical site is not flat and level, such as a bent knee, a bent elbow, or the back or chest of a patient lying on his side.

The shape and size of any embodiment may be designed to fit the intended surgical site or function of the embodiment. Correctly shaping and sizing the embodiment results in enhanced control and distribution of the therapeutic material and reduced waste.

Any embodiment may be enhanced with a port enabling the embodiment to be filled, refilled, or flushed. The flow through the port may be controlled by a valve. The port may be terminated with a standard tapered Luer fitting or flexible valve such as those described in co-owned International Application No. PCT/US2012/053302.

Any embodiment enhanced with the port may be further enhanced with multiple liquid paths that help to control and distribute liquid evenly.

Any embodiment may be designed such that a sealed interior region may be supported central of its sealed perimeter by sealing the upper and lower layers, without substantially partitioning the interior region into separate interior regions.

The layer materials may be made thicker or thinner to provide facilitate incision. The layer materials may also be varied to facilitate incision. The layer materials may also be varied to allow for a clean cut of the material that does not fray or flake, in order to minimize introducing the layer material itself into the incision.

The therapeutic material may be an antimicrobial, a lubricant, a tissue moisturizer, an anesthetic, or any combination thereof. Furthermore the viscosity of the material may be varied to control the flow into and coating of the incision. The volume of the therapeutic material may also be varied in order to provide the correct dose to the patient.

The above summary of the various embodiments of the invention is not intended to describe each illustrated embodiment or every implementation of the invention. This summery represents a simplified overview of certain aspects of the invention to facilitate a basic understanding of the invention and is not intended to identify key or critical elements of the invention or delineate the scope of the invention.

“Flexible” refers to being able to be bent around a rod of diameter 10 cm, preferably 2 cm, more preferably 1 or 2 mm, most preferably 0.25 mm or less;

“Flowable liquid” refers to a composition that takes the shape of a container in which it is placed within a period of 4 hours.

“Impermeable” refers to having essentially no air flow (less than 1 centimeter per minute from a 100 square centimeter area) or noticeable holes such as pin holes when pressurized with 0.5 psig in a suitable device that prevents distortion such as a supported sheet that downstream exits under water to detect air flow or volume changes;

“Incise drape” refers to a sterile adhesive-coated barrier film intended to cover the target incision site of the patient (and surrounding area), through which a surgeon makes an incision to access a body cavity; the incise drape prevents the migration of flora from the surface of the skin into the surgical site;

“Liquid-impervious” refers to being able to hold out a sealed column of water of at least 20 inches according to AATCC 127-2008 Water Resistance: Hydrostatic Pressure Test; thin or elastics samples that may distort in the test should be properly supported to prevent distortion; a liquid-impervious film layer may or may not be impermeable to vapor;

“Pouch” refers to a flexible bag, pocket, enclosure, reservoir, package, cavity, or vessel made of a film or films that preferably are inert to materials within it and impervious to liquids in the surrounding environment; preferably it is of a unitary construction, although a combination of compatible materials (and layers) can be used;

By “transparent” it is meant that when the filled incise drape of the present disclosure is applied to a patient (e.g., at an intended operation site), the area underlying the filled incise drape can be visualized sufficiently to permit observation of the intended operation site by a health care worker.

“Unitary construction” means of one material.

The terms “comprises” and variations thereof do not have a limiting meaning where these terms appear in the description and claims.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

As used herein, “a,” “an,” “the,” “at least one,” and “one or more” are used interchangeably. Thus, for example, a stress-distributing layer comprising a water-absorbing polymer can be interpreted to mean that the stress-distributing layer includes “one or more” water-absorbing materials.

As used herein, the term “or” is generally employed in its sense including “and/or” unless the content clearly dictates otherwise.

The term “and/or” means one or all of the listed elements or a combination of any two or more of the listed elements (e.g., preventing and/or treating an affliction means preventing, treating, or both treating and preventing further afflictions).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF DRAWINGS

FIGS. 1a, 1b, and 1c are exemplary embodiments of a filled incise drape of the present disclosure;

FIGS. 2a to 2f show an exemplary embodiment of a method of using a filled incise drape of the present disclosure;

FIGS. 3a 3b are perspective and cutaway profile views, respectively, of an exemplary embodiment of a filled incise drape of the present disclosure;

FIGS. 4a to 4c are perspective, plan, and cutaway profile views, respectively, of an exemplary embodiment of a filled incise drape of the present disclosure;

FIGS. 5a and 5b are plan and cutaway profile views, respectively, showing an exemplary embodiment of a plurality of filled incise drapes of the present disclosure;

FIGS. 6a to 6f are views of exemplary embodiments of a filled incise drape of the present disclosure that include a surgical device;

FIGS. 7a to 7c are perspective, plan, and cutaway profile views, respectively, of an exemplary embodiment of a filled incise drape of the present disclosure;

FIGS. 8 and 9 are cutaway profile views showing performance of exemplary embodiments of filled incise drapes of the present disclosure upon bending;

FIGS. 10a to 10g are views of exemplary embodiments of filled incise drapes of the present disclosure;

FIGS. 11a and 11b are perspective and cutaway profile views, respectively, of an exemplary embodiment of a filled incise drape of the present disclosure;

FIGS. 12a, 12b, and 12c are perspective, exploded perspective, and cutaway profile views, respectively, of an exemplary embodiment of a filled incise drape of the present disclosure, and FIG. 12d is a profile view showing performance of the same filled incise drape upon bending;

FIGS. 13a, 13b, 13c, and 13d are perspective, plan, and two different cutaway profile views, respectively, of an exemplary embodiment of a filled incise drape of the present disclosure.

FIG. 14 is a cutaway profile view of an exemplary embodiment of a filled incise drape of the present disclosure.

FIG. 15 is a cutaway profile view of an exemplary embodiment of a filled incise drape of the present disclosure.

### DETAILED DESCRIPTION

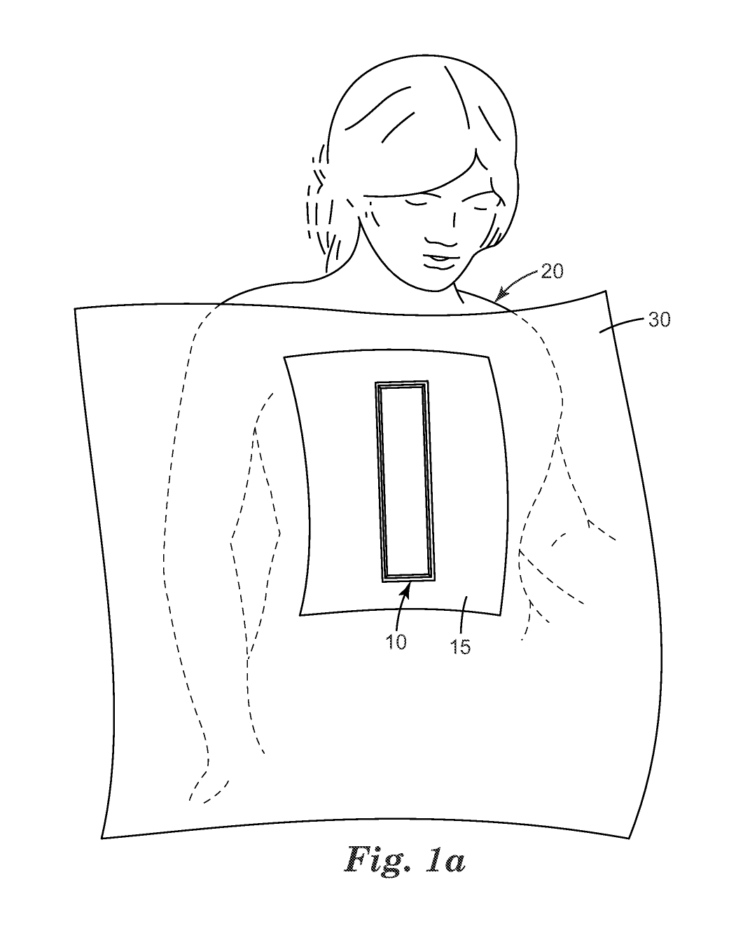

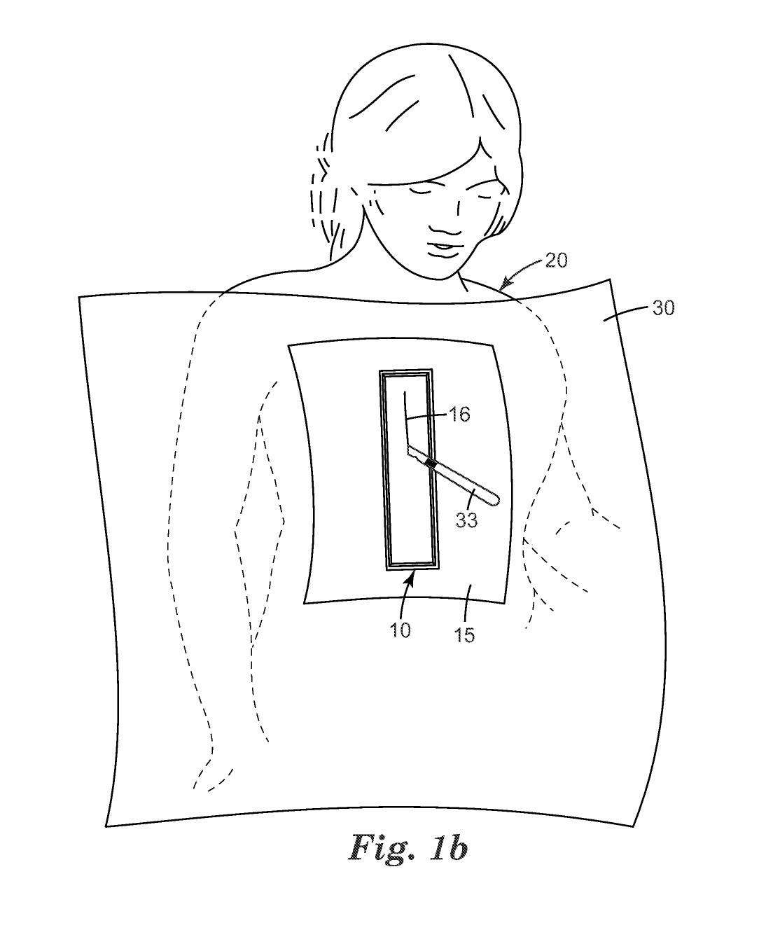

Presented in FIGS. 1a, 1b, and 1c are exemplary embodiments of a filled incise drape 10 of the present disclosure. Filled incise drape 10 is a therapeutic-material filled surgical incise drape. In FIGS. 1a and 1b, filled incise drape 10 is secured to the top surface of a traditional incise drape 15. Filled incise drape 10 may be secured to traditional incise drape 15 by, for example, being heat sealed or adhesively bonded, or both. In FIGS. 1a and 1b, a larger surgical drape 30 is also shown. In FIG. 1c, filled incise drape 10 is adhered directly to a patient's skin 20. Skin 20 includes underlying tissue. In each of FIGS. 1a to 1c, filled incise drape 10 is positioned for making an incision 16 through filled incise drape 10 and into patient's skin 20. Upon incising, and through the incisions made through filled incise drape 10, the therapeutic material (not shown) flows into the surgical site, at least temporarily filling at least a portion of the incision 16, and coating at least a portion of the wound, the wound edge, the scalpel 33, and any surgical tools (not shown) that might pass there through. In one particularly preferred method a shallow incision (e.g. 1 to 10 mm, preferably 2-6 mm, more preferably 2-4 mm) is initially made in the skin allowing the therapeutic material to essentially completely fill the incision, changing the scalpel blade to ensure sterility, and allowing the antiseptic to kill bacteria in the incision for a period of time (e.g. at least 15 seconds, preferably at least 30 seconds, and most preferably at least 45 seconds) before incising further. In embodiments where the therapeutic material is a rapid acting antiseptic, once delivered it may then begin to protect the patient from a surgical site infection.

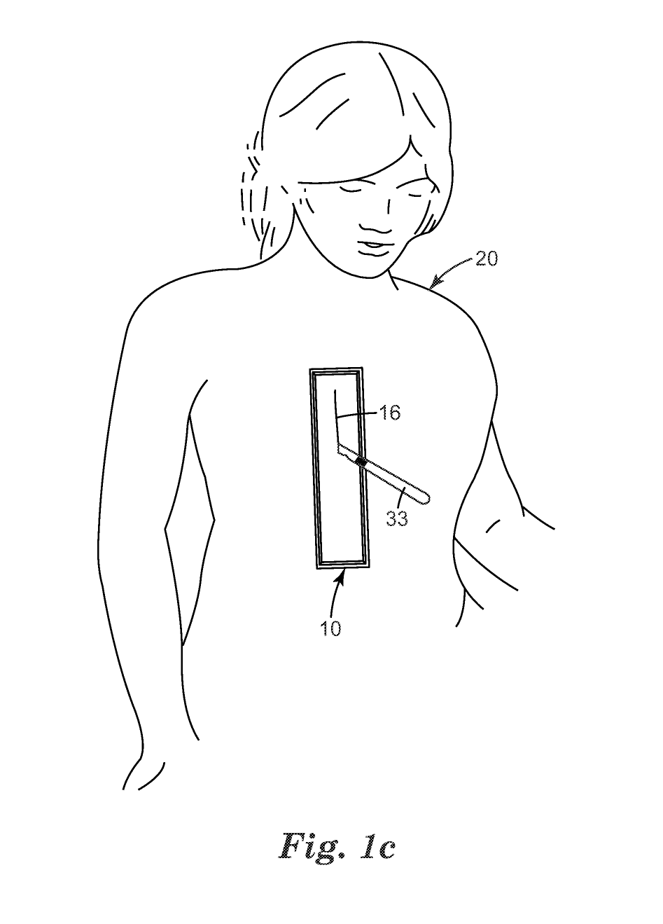

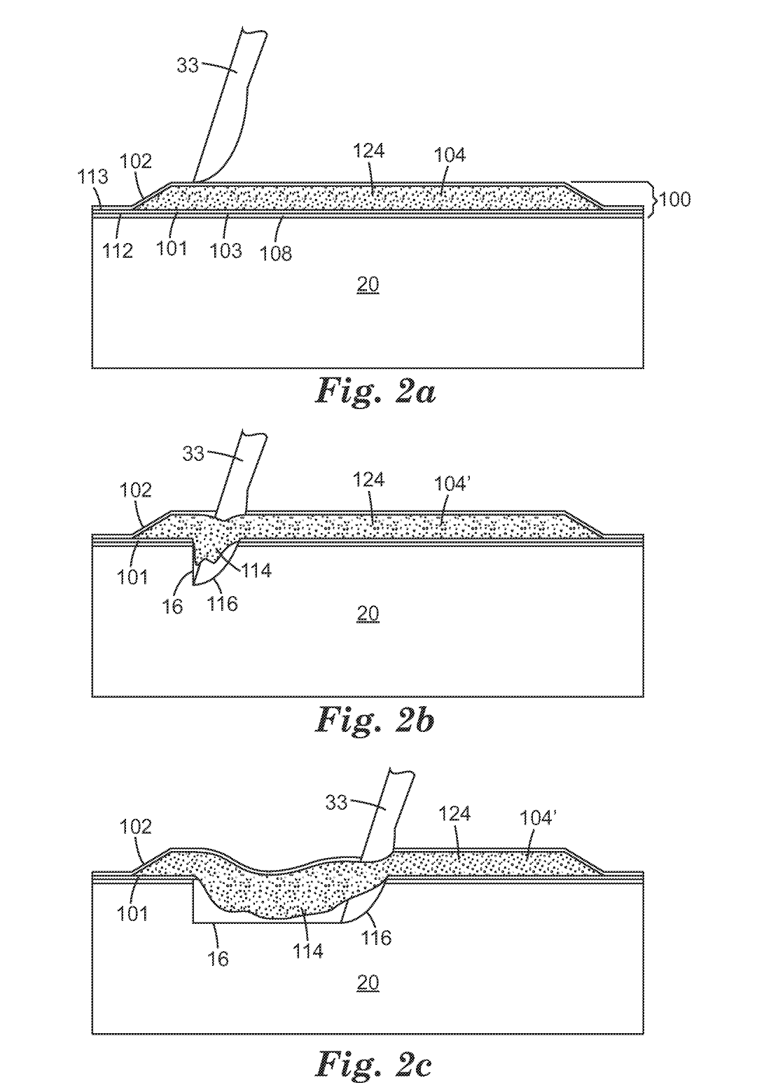

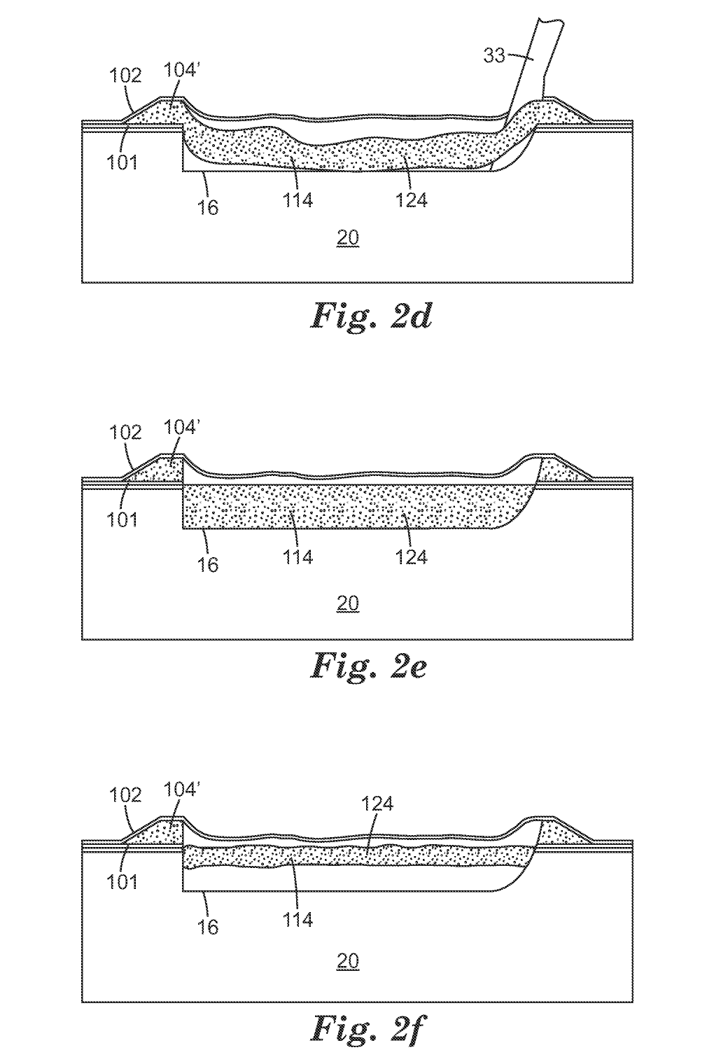

Presented in FIGS. 2a to 2f are profile section views of an exemplary embodiment of a liquid-filled surgical incise drape 100 of the present disclosure, demonstrating a process of, and at least one variation of, delivering the liquid to the surgical site.

In the exemplary embodiment shown in FIG. 2a, filled incise drape 100 is depicted having an second film layer 102, a first film layer 101, wherein second film layer 102 and first film layer 101 are sealed together to form at least one interior region 104 for enclosing a liquid, and an adhesive layer 108 disposed on exterior surface 103 of first film layer 101. Interior region 104 is a liquid reservoir, including sealed perimeter region 113 where upper layer 102 and first film layer 101 are shown sealed together. In FIG. 2a, the at least one interior region 104 is shown as filled with liquid 124, which in a typical embodiment is a therapeutic material, although liquids other than therapeutic materials are also contemplated (e.g., lubricants). First film layer 101 and adhesive layer 108 disposed thereon form a composite layer that is liquid-impervious, and therefore liquid therapeutic material does not pass from interior region 104 to skin 20 until an incision or puncture of the composite layer is made.

In FIG. 2a, filled incise drape 100 is shown affixed to a patient's skin 20 with adhesive layer 108, which is shown as a continuous layer on exterior surface 103 of first film layer 101. However, other embodiments are also contemplated, for example, having adhesive layer 108 disposed on periphery 112 of exterior surface 103 of first film layer 101, although typically it may be desirable for adhesive layer 108 to cover most or all of to cover most or all of exterior surface 103 of first film layer 101.

In an embodiment, the incise drape 100 may be affixed to the skin 20, not by applying adhesive layer 108 first to the filled incise drape, but instead by applying adhesive layer 108 or other reactive substance (e.g., a substance that will bond to the exterior surface 103 of first film layer 101) first to the skin of the patient 20, or to an intermediary incise drape or surgical drape, and subsequently positioning exterior surface 103 of first film layer 101 on adhesive layer 108.

In FIG. 2b, incise drape 100 is shown having been incised with a scalpel 33 that has cut through second film layer 102, also through therapeutic material 124 of interior region 104, and through first film layer 101 and has created an incision 16 having leading edge 116 in the skin (i.e., tissue) 20. As scalpel 33 has cut through liquid-impervious layer 101, therapeutic material 124 has been released from reservoir 104′ (interior region 104, after being incised, is referred to here as reservoir 104′) and is shown flowing in the location identified with 114 into incision 16. Therapeutic material 124 there delivered 114 may then begin to help disinfect the incision site of the patient.

In FIG. 2c, scalpel 33 is shown to have advanced leading edge 116 of incision 16, thereby extending the incisions made in second film layer 102 and first film layer 101, and thereby delivering more therapeutic material 124 from reservoir 104′ into incision 16. Therapeutic material 124 there delivered 114 may then begin to disinfect the incision site to protect the patient from a surgical site infection.

In FIG. 2d, scalpel 33 is shown to have advanced the leading edge 116 of incision 16, thereby extending the incisions made in second film layer 102 and first film layer 101, thereby delivering more therapeutic material 124 from reservoir 104′ into incision 16. Therapeutic material 124 there delivered 114 may then begin to disinfect the incision site to protect the patient from a surgical site infection.

In FIG. 2e, the incision 16 has been completed and scalpel 33 has been removed from incision 16. Additionally, therapeutic material 124 there delivered 114 has left a nearly empty reservoir 104′ and has settled within the surgical site. Importantly, the therapeutic material has coated at least a portion of the skin incision and preferably coats the entire length of the incision, as shown.

In FIG. 2f, the volume and properties (e.g., viscosity) of therapeutic material 124 have been controlled and/or altered such that the therapeutic material there delivered 114 does not flow all the way into the incision 16, but instead remains within the demonstrated zone. This is especially important because such controlled delivery of the therapeutic material would allow for limited systemic exposure of the topical therapeutic composition, for example, limiting the flow of therapeutic material 124 to the skin layers and immediate subcutaneous region but not deeper into the body cavity. In some embodiments, a suitable volume of therapeutic material in the filled incise drape can be in a range of about 100 microliters to about 10 milliliters, or more preferably in a range of 500 microliters to 2 milliliters (for example, in a surgical incise drape having a length of about 7.5 centimeters by about 5 centimeters). These ranges of volumes can be useful, for example, for limiting the amount of therapeutic material released as the incision is being made. By also adjusting the viscosity of the therapeutic material, penetration of the therapeutic material into skin at the incision site can be at least partially controlled.

In some embodiments it can be advantageous to minimize a thickness of the interior region filled with therapeutic material, for example, to permit maximal sensing of underlying tissue and structures by touch prior to making an incision, as well as permitting optimal viewing through the filled incise drape.

In typical embodiments, second film layer 102 comprises a liquid-impervious and flexible film, which may be of the same material as first film layer 101, or may be selected to have another type of material; typically, however, second film layer 102 is selected to be liquid-impervious and flexible.

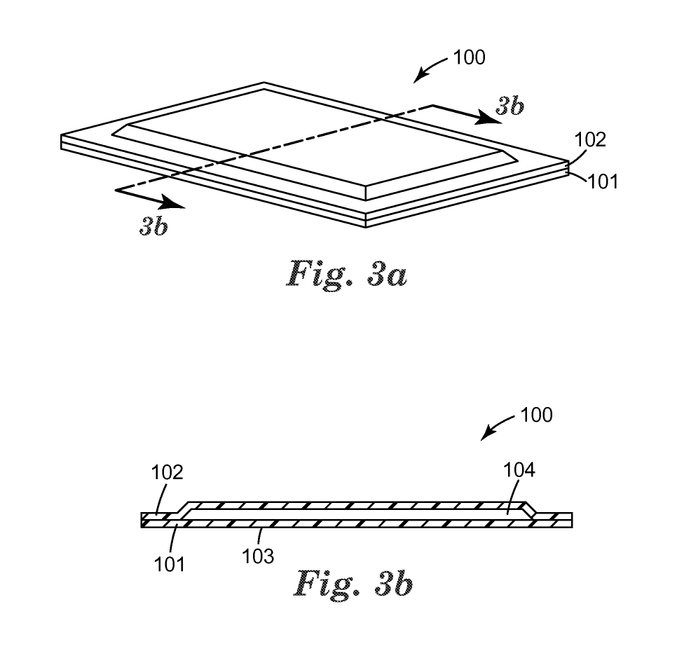

FIGS. 3a and 3b are perspective and profile views, respectively, of an exemplary embodiment of filled incise drape 100 of the present disclosure. Filled incise drape 100 is comprised of second film layer 102 and first film layer 101, wherein second film layer 102 and first film layer 101 are sealed together to form at least one interior region 104 having a sealed periphery, and an adhesive layer (not shown) disposed on exterior surface 103 of first film layer 101. In typical embodiments, at least one interior region 104 is liquid-filled, preferably with a therapeutic material. Filled incise drape 100 may be adhered to the skin of a patient (not shown) by the adhesive coated exterior surface 103 and an incision may be made there-through. Upon making the incision (not shown) the therapeutic material filling at least one interior region 104 will be released into the incision. When the therapeutic material is an antiseptic or antibiotic, this would result in killing bacteria present in the incision or within the surgical site.

In an alternative embodiment, layers 101 and 102 could be of unitary construction, such as the opposite walls of a flattened tube.

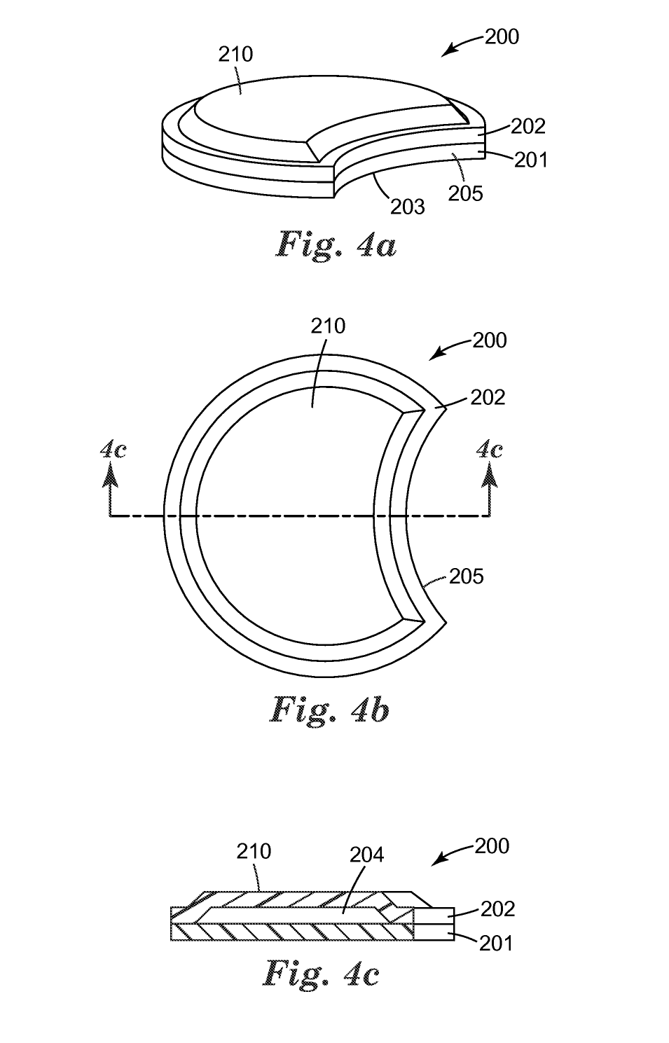

Presented in FIGS. 4a-4c is another embodiment of a filled incise drape 200 of the present disclosure. Filled incise drape 200 is comprised of second film layer 202 (having exterior surface 210) and first film layer 201, wherein second film layer 202 and first film layer 201 are sealed together to form at least one interior region 204 having a sealed periphery, and an adhesive layer (not shown) disposed on exterior surface 203 of first film layer 201. In typical embodiments, at least one interior region 204 is liquid-filled, preferably with a therapeutic material. Filled incise drape 200 may be adhered to the skin of a patient (not shown) by the adhesive layer disposed on exterior surface 203, and an incision may be made there-through. Upon making the incision (not shown) the therapeutic material filling at least one interior region 204 will be released into the incision. When the therapeutic material is an antiseptic or antibiotic, this would result in killing bacteria present in the incision or within the surgical site.

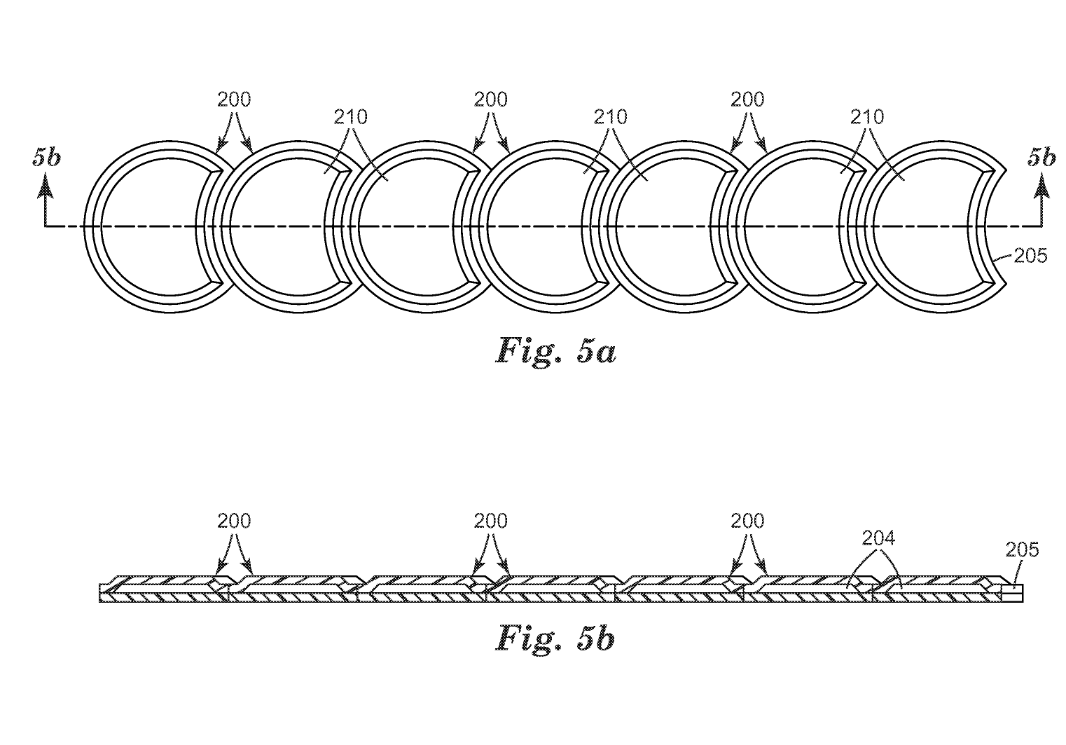

In the plan view of filled incise drape 200 shown in FIG. 4b, filled incise drape 200 has a generally circular shape, and a cut-out portion 205. Cut-out portion 205 has a radius similar to the radius of the circular shape, so that filled incise drapes 200 may be positioned proximately to nearly continuously span an incision that is longer than can be covered by a single filled incision drape. FIGS. 5a-5b shows an exemplary embodiment of the present disclosure wherein a plurality of filled incise drapes 200 may be placed adjacent to each other on the same surgical drape (not shown), so that the length of the incision (not shown) is spanned. The combination of circular shape and cut-out portion 205 allows for flexibility in the placement of a plurality of filled incise drapes 200, to accommodate a variety of surgical incisions.

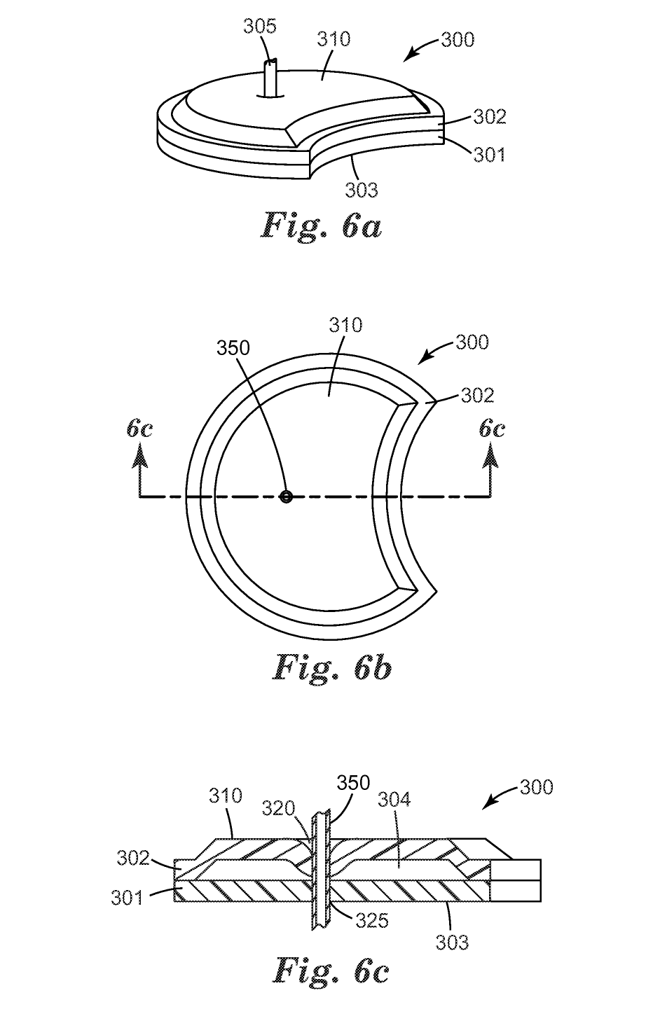

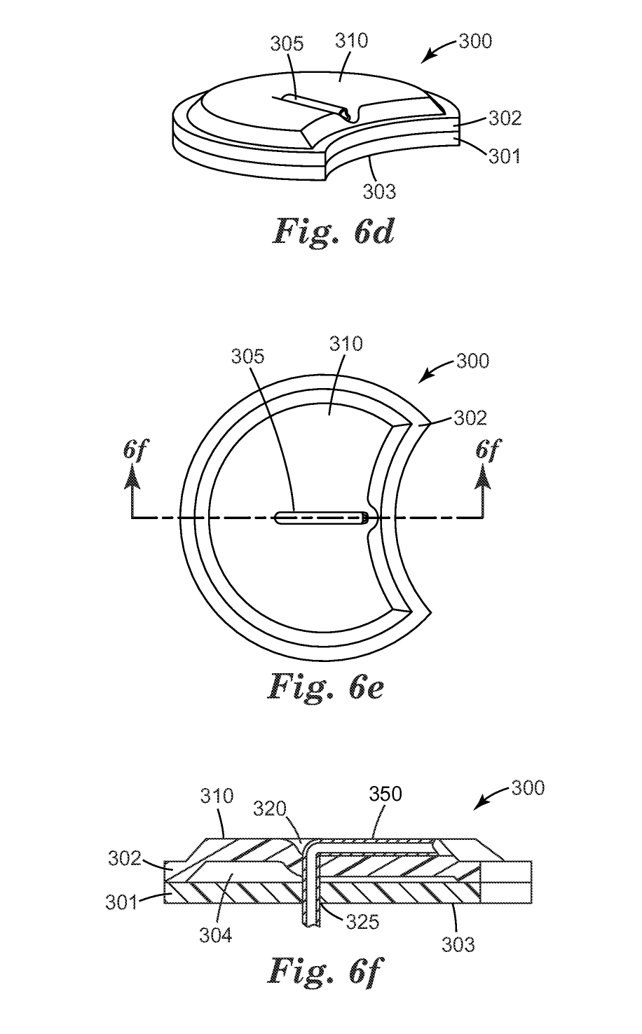

Presented in FIGS. 6a, 6b, and 6c is an exemplary embodiment of a filled incise drape 300 of the present disclosure. Filled incise drape 300 is comprised of second film layer 302 (having exterior surface 310) and first film layer 301, wherein second film layer 302 and first film layer 301 are sealed together to form at least one interior region 304 having a sealed periphery, and an adhesive layer (not shown) disposed on exterior surface 303 of first film layer 301. The adhesive may optionally contain an antimicrobial agent disposed therein or thereon such as disclosed in U.S. Pat. Nos. 4,323,557, 4,310,509, and U.S. Published Patent Application No. 2006/0035039. In typical embodiments, at least one interior region 304 is liquid-filled, preferably with a therapeutic material. Filled incise drape 300 may be adhered to the skin of a patient (not shown) by the adhesive coated exterior surface 303 and a surgical device 305 may be implanted percutaneously there-through, for example, at entry point 320 and exit point 325. Upon implanting the surgical device 305 there-through, therapeutic material filling 304 will be released into the wound site (not shown) killing bacteria present the wound site. Furthermore, therapeutic material will prevent any bacteria from migrating down the surgical device into the wound site (not shown). Finally, as presented in the exemplary embodiments shown in FIGS. 6d to 6f, surgical device 305 can be bent over to lay along the surface of second film layer 302. In some embodiments, surgical device 305 can be secured to filled incise drape 300 with an adhesive covering (e.g., that adhesive covering available from 3M Co., St. Paul, Minn., under the trade designation “3M TEGADERM”)(not shown) or tape. Such an adhesive covering or tape would prevent excess therapeutic material from migrating out of or away from filled incise drape 300.

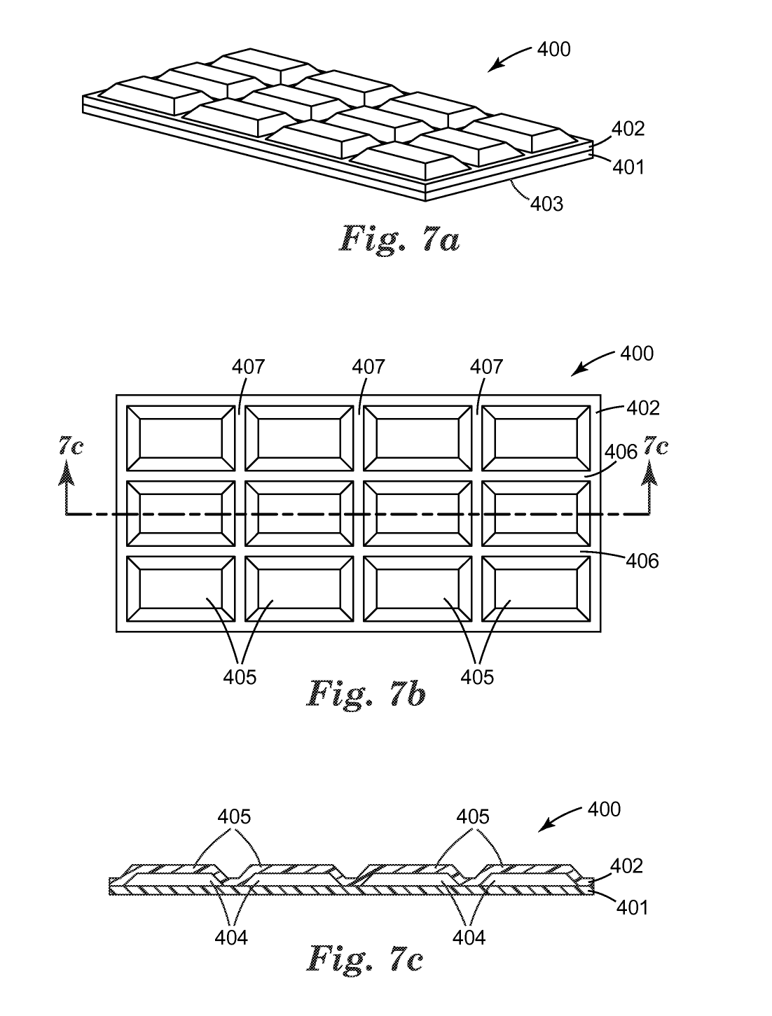

FIGS. 7a to 7c are views of an exemplary embodiment of a filled incise drape 400 of the present disclosure. Filled incise drape 400 is comprised of second film layer 402 and first film layer 401, wherein second film layer 402 and first film layer 401 are sealed together to form at least one interior region 404 having a sealed periphery, and an adhesive layer (not shown) disposed on exterior surface 403 of first film layer 401. In typical embodiments, at least one interior region 404 is liquid-filled, preferably with a therapeutic material. Filled incise drape 400 may be adhered to the skin of a patient (not shown) by the adhesive coated exterior surface 403 and an incision may be made there-through. Upon making the incision, the (e.g., therapeutic material) filling of at least one interior region 404 will be released into the incision (not shown) killing bacteria present in the incision or within the surgical site. In this particular embodiment, individual liquid-filled pouches 405 are independently sealed by additional horizontal seals 406 and additional vertical seals 407. This is especially useful when the surgical site is not flat and level, such as a bent knee, a bent elbow, or the back or chest of a patient lying on his side.

In some embodiments of a filled incise drape having a plurality of interior regions, therapeutic material may be separated in different interior regions (e.g., a plurality of interior regions 404 shown in FIG. 7c), and a first therapeutic material in a first interior region can be selected to be the same as or different from a second therapeutic material in a second interior region. It may be useful to introduce the first therapeutic material while making the first incision, but the second therapeutic material (and, optionally, further therapeutic materials) can be introduced at a later time, perhaps in the middle of surgery or prior to closing the incision. The viscosity of the therapeutic materials in each of the interior regions can usefully be selected to be different from each other, for example it the first liquid can be more viscous to allow coating of skin surfaces during an initial incision, and subsequent therapeutic materials can have lower viscosity, to allow for deeper into the incision area. It is also possible to introduce antiseptic therapeutic materials at different concentrations and at different times, for example, to limit toxicity.

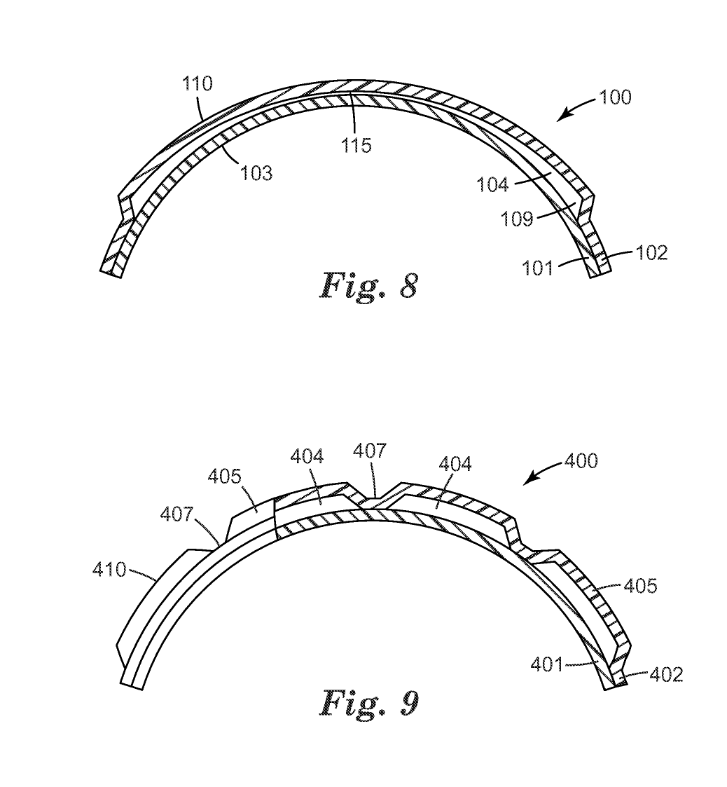

FIG. 8 illustrates how drape 100, when significantly bent, might narrow in middle section 108, and not narrow significantly at edges 109. This narrowing could cause the amount of the therapeutic material filling at least one interior region 104 to be released in a variable manner along the length of the incision (not shown), which may be undesirable. Middle section 115 may narrow due to varying elasticity of the materials in second film layer 102 and first film layer 101. FIG. 9 illustrates how seals 407 allow for filled incise drape 400 to bend and for liquid-filled pouches 405 to maintain a constant thickness so that when an incision (not shown) is made there-through, an even amount of therapeutic material is released along the length of the incision (not shown).

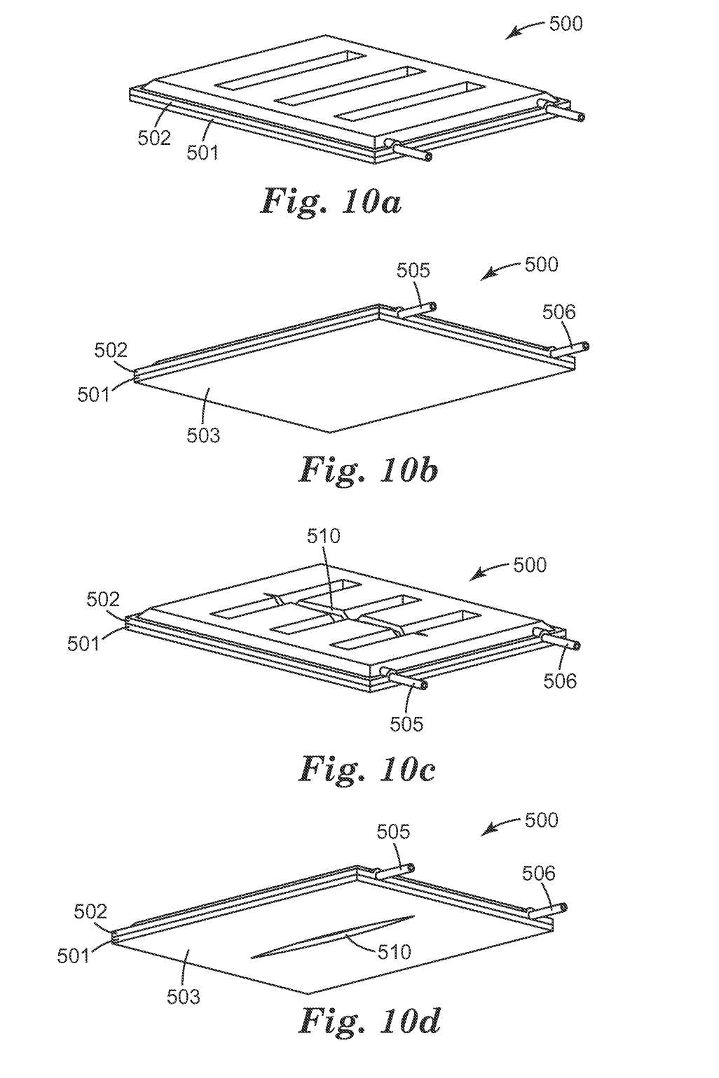

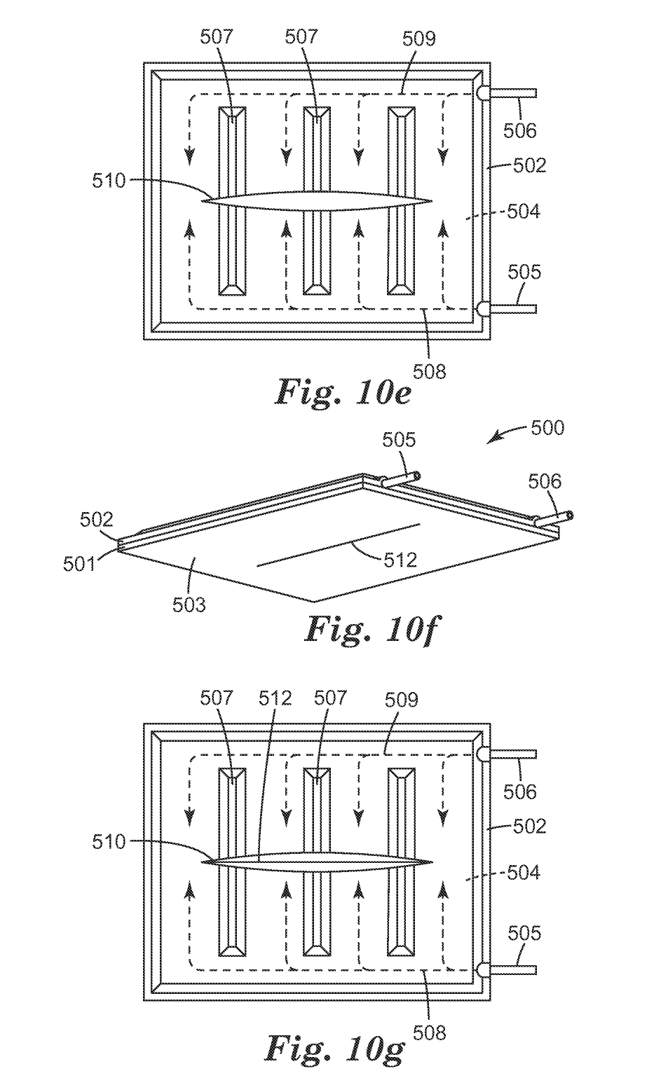

FIGS. 10a to 10g illustrate further exemplary embodiments of a filled incise drape 500 of the present disclosure. Filled incise drape 500 is comprised of second film layer 502 and first film layer 501, wherein second film layer 502 and first film layer 501 are sealed together to form at least one interior region 504 for enclosing a liquid, and an adhesive layer (not shown) disposed on exterior surface 503 of first film layer 501. In typical embodiments, at least one interior region 504 is liquid-filled, preferably with a therapeutic material. Filled incise drape 500 may be adhered to the skin of a patient (not shown) by the adhesive coated exterior surface 503 and an incision may be made there-through. Upon making the incision through filled incise drape 500 and into the patient's skin, the therapeutic material will be released from at least one interior region 504 into the incision (not shown) killing bacteria present in the incision or within the surgical site. In this particular embodiment, ports 505 and 506 are affixed to the dressing in fluid communication with fluid pathways 508 and 509 established by seals 507 and by the incision 510 (as shown, for example, in FIGS. 10e and 10g). In some embodiments, no liquid will move (unless there is a pressure gradient between the ports 505 and 506) until filled incise drape 500 is incised there-through. In such case that filled incise drape 500 is incised there-through, liquid will flow through the ports 505 and 506 and follow the fluid pathways 508 and 509 into the incision 510, respectively.

FIGS. 10f and 10g illustrate an exemplary embodiment of filled incise drape 500 wherein second film layer 502 is in tension, so that as it is incised, upper incision 511 through second film layer 502 immediately opens to allow a view for making lower incision 512 through first film layer 501. Such performance of a filled incise drape 500 will provide the surgeon with a clear view for making lower incision 512 through the skin-side layer 501, which might be at least partially obscured in other embodiments.

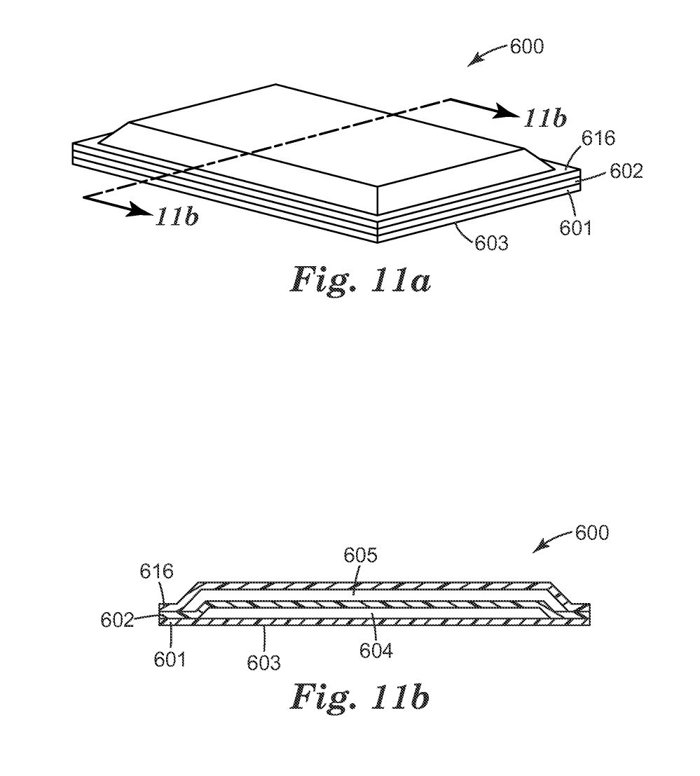

FIGS. 11a and 11b illustrate an exemplary embodiment of a filled incise drape 600 of the present disclosure. Filled incise drape 600 is comprised of second film layer 602 and first film layer 601, wherein second film layer 602 and first film layer 601 are sealed together to form at least one interior region 604 having a sealed periphery, and an adhesive layer (not shown) disposed on exterior surface 603 of first film layer 601. Filled incise drape 600 further comprises a cap layer 616, wherein cap layer 616 and second film layer 602 are sealed together to form at least one interior region 605 having a sealed periphery. In typical embodiments, at least one interior region 604 and at least one interior region 605 are each liquid-filled, preferably with a therapeutic material, although the liquid in at least one interior region 604 and the liquid in at least one interior region 605 may be selected to be different therapeutic materials. Filled incise drape 600 may be adhered to the skin of a patient (not shown) by the adhesive coated exterior surface 103 and an incision may be made there-through. Upon making the incision (not shown) the therapeutic material fillings 604 and 605 will be released into the incision (not shown) killing bacteria present in the incision or within the surgical site. The addition of additional cap layers to establish additional material fillings may be contemplated. The cap layer is typically selected to be liquid-impervious and flexible.

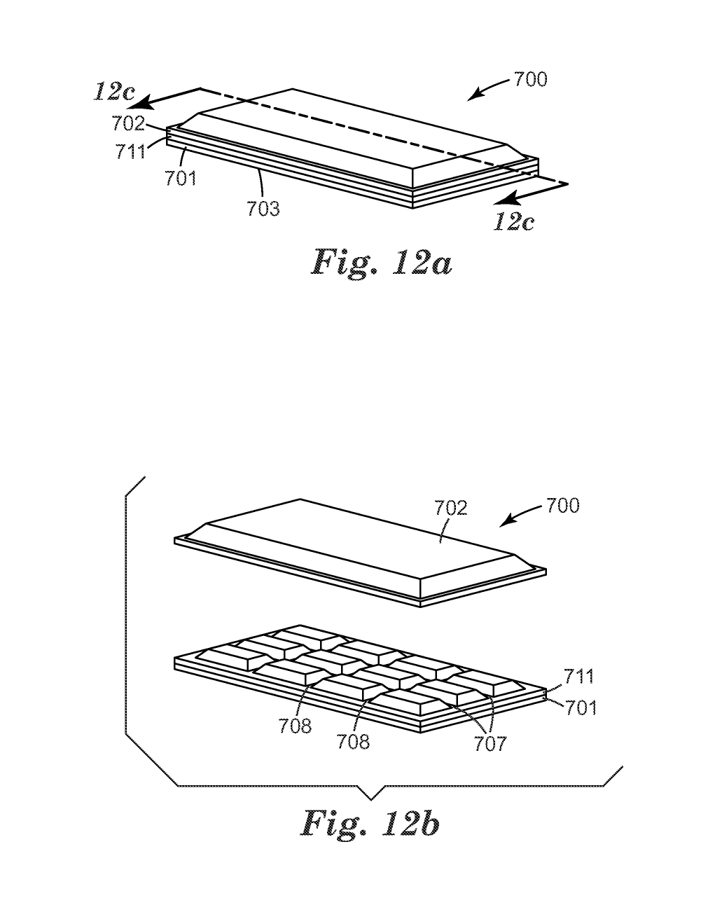

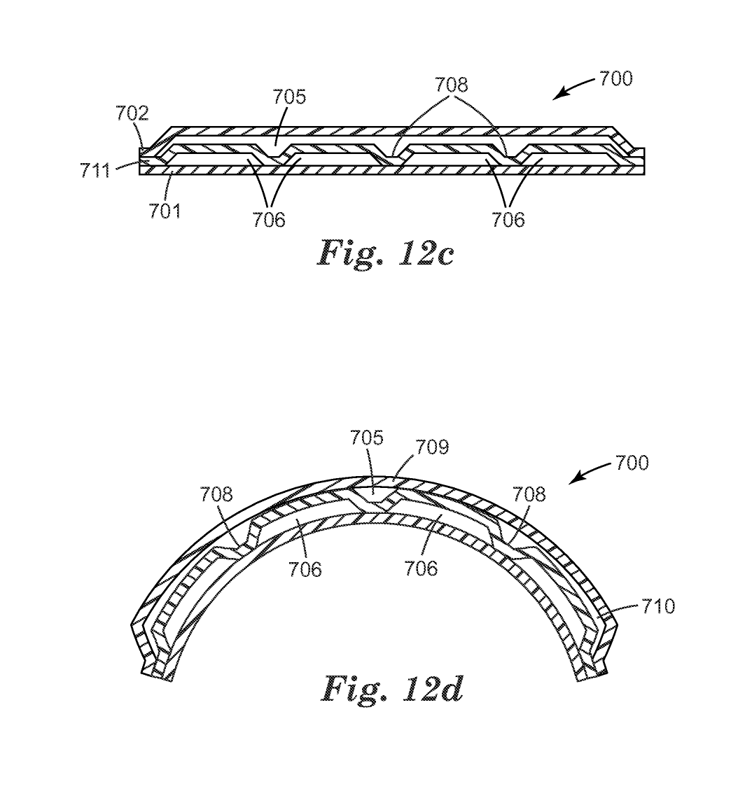

FIGS. 12a to 12c illustrate an exemplary embodiment of a filled incise drape 700 of the present disclosure. Filled incise drape 700 is comprised of second film layer 702, an intermediate film layer 711, first film layer 701, and an adhesive layer (not shown) disposed on exterior surface 703 of first film layer 701, wherein second film layer 702 and intermediate film layer 711 are sealed together to form at least one interior region 705 having a sealed periphery, and wherein intermediate film layer 711 and first film layer 701 are sealed together to form a plurality of interior regions 706. The at least one interior region 705 can be liquid-filled, and typically the liquid is selected to be a therapeutic material. Filled incise drape 700 may be adhered to the skin of a patient (not shown) by the adhesive coated exterior surface 703 and an incision may be made there-through. Upon making the incision (not shown), therapeutic material will be released from at least one interior region 105 into the incision (not shown) killing bacteria present in the incision or within the surgical site. In this particular embodiment, individual supporting pouches 706 are discretely sealed by additional horizontal seals 707 and additional vertical seals 708. This is especially useful when the surgical site is not flat and level, such as a bent knee, a bent elbow, or the back or chest of a patient lying on his side. FIG. 12d demonstrates how a filled incise drape 700 with these supporting features, when significantly bent, might narrow only slightly, or not at all, in the middle section 709 when compared to the edges 710, resulting in a more even release of the amount of the therapeutic material along the length of the incision.

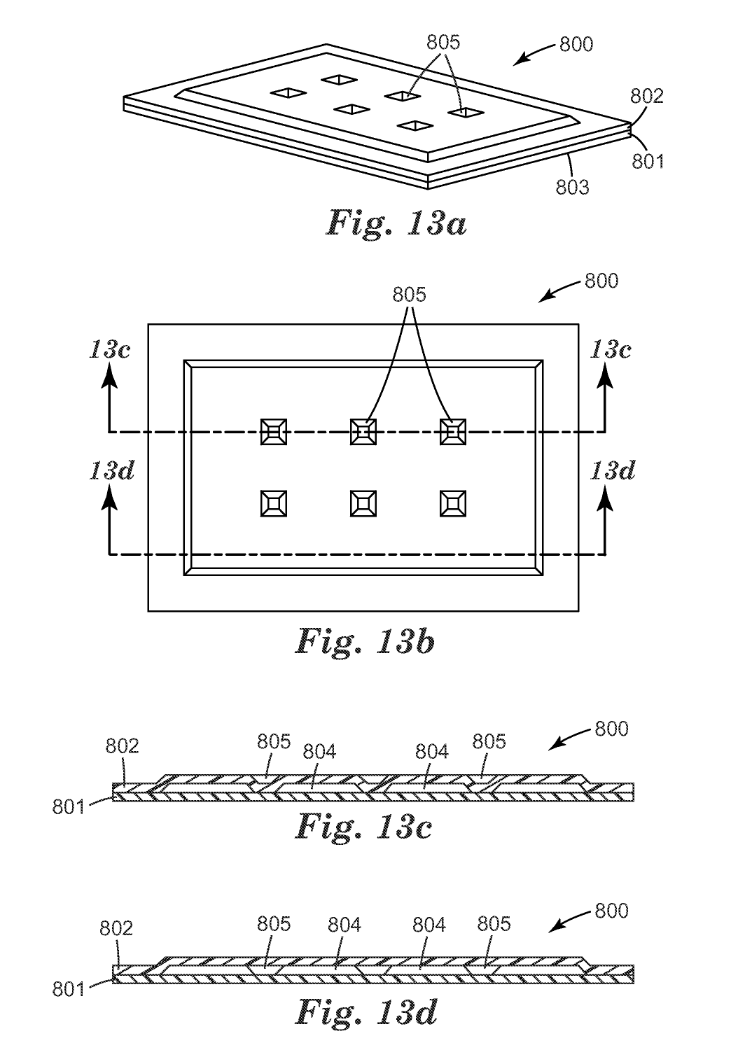

FIGS. 13a to 13d illustrate an exemplary embodiment of a filled incise drape 800 of the present disclosure. Filled incise drape 800 is comprised of second film layer 802 and first film layer 801, wherein second film layer 802 and first film layer 801 are sealed together to form at least one interior region 804 having a sealed periphery, and an adhesive layer (not shown) disposed on exterior surface 803 of first film layer 801. In typical embodiments, at least one interior region 804 is liquid-filled, preferably with a therapeutic material. Filled incise drape 800 may be adhered to the skin of a patient (not shown) by the adhesive coated exterior surface 803 and an incision may be made there-through. Upon making the incision (not shown) the therapeutic material filling at least one interior region 104 will be released into the incision. When the therapeutic material is an antiseptic or antibiotic, this would result in killing bacteria present in the incision or within the surgical site. In this particular embodiment, spot seals 805 seal second film layer 802 to first film layer 801 in spots within at least one interior region 804, without creating discrete liquid voids or pouches. Thus filled incise drape 800 can maintain contents under pressure without bulging in the middle. Further, this configuration of filled incise drape 800 could help to more evenly control the amount of the therapeutic material released along the length of the incision. This is especially useful when the surgical site is not flat and level, such as a bent knee, a bent elbow, or the back or chest of a patient lying on his side.

FIG. 14 is a profile view of an exemplary embodiment of filled incise drape 900 of the present disclosure. Filled incise drape 900 is comprised of formed layer 902 and release liner layer 910, wherein formed layer 902 and release liner layer 910 are releasably sealed together to form at least one interior region 904 having a sealed periphery, and an adhesive 908 layer disposed between formed layer 902 and release liner layer 910. In the embodiment shown, a releasable (but not resealable) adhesive 907 (or seal) is also disposed between formed layer 902 and release liner layer 910, included, for example, to facilitate sealing formed layer 902 and release liner layer 910 together until use. Adhesive 908 may be selected to be more adherent, or less adherent, than adhesive 907, to create the appropriate sealing function, and also for material compatibility with filling 924. Also, adhesive 908 is shown as being on the interior relative to adhesive 907; in other embodiments, adhesive 908 can be exterior relative to adhesive 907. In typical embodiments, at least one interior region 904 is liquid-filled, preferably with a therapeutic material. Once release liner 910 has been removed, filled incise drape 900 may be adhered to the skin of a patient (not shown) by adhesive 908, and an incision may be made there-through. Upon making the incision (not shown) therapeutic material 924 filling at least one interior region 904 will be released into the incision. When therapeutic material 924 is an antiseptic or antibiotic, this would result in killing bacteria present in the incision or within the surgical site.

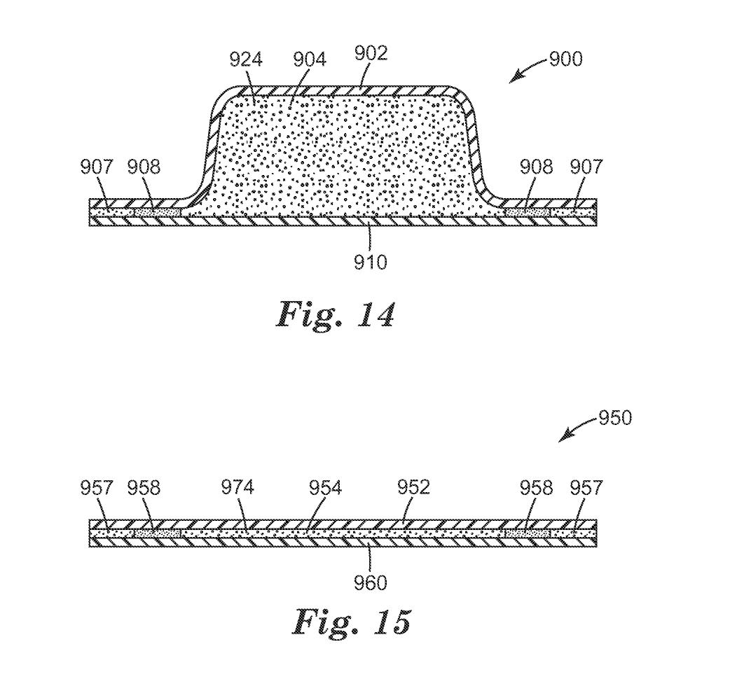

FIG. 15 is a profile view of an exemplary embodiment of filled incise drape 950 of the present disclosure. Filled incise drape 950 is comprised of flexible film layer 952 and release liner layer 960, wherein flexible film layer 952 and release liner layer 960 are releasably sealed together to form at least one interior region 954 having a sealed periphery, and an adhesive 958 layer disposed between flexible film layer 952 and release liner layer 960. In the embodiment shown, a releasable (but not resealable) adhesive 957 (or seal) is also disposed between flexible film layer 952 and release liner layer 960, included, for example, to facilitate sealing flexible film layer 952 and release liner layer 960 together until use. Adhesive 958 may be selected to be more adherent, or less adherent, than adhesive 957, to create the appropriate sealing function, and also for material compatibility with filling 974. Also, adhesive 958 is shown as being on the interior relative to adhesive 957; in other embodiments, adhesive 958 can be exterior relative to adhesive 957. In typical embodiments, at least one interior region 904 is liquid-filled, preferably with a therapeutic material. Once release liner 960 has been removed, filled incise drape 950 may be adhered to the skin of a patient (not shown) by adhesive 958, and an incision may be made there-through. Upon making the incision (not shown) therapeutic material 974 filling at least one interior region 954 will be released into the incision. When therapeutic material 974 is an antiseptic or antibiotic, this would result in killing bacteria present in the incision or within the surgical site.

For the embodiments of a filled incise drape shown in FIGS. 14 and 15, the therapeutic materials (924 and 974, respectively), is formulated to be substantially viscous, such that it is controllable without a skin-side layer.

Embodiments of a filled incise drape are contemplated wherein the enclosure is hermetically sealed, or not hermetically sealed but included within an overall package that is hermetically sealed (i.e., wherein the filled incise drape sealed in an air-tight package).

Articles of the present disclosure can be made by any suitable method, including standard packaging methods that use form, fill and seal equipment. In one embodiment, the process includes: (1) bringing together 1 or 2 films and thermally sealing to form a vertically-oriented tube; (2) sealing across a bottom portion of the tube; (3) filling a portion of the tube (above the bottom portion) with liquid; sealing across a top portion of the tube; and optionally (5) heat- or ultrasonically-seal an emboss pattern on the portion of the tube between the seal across the bottom and top portions of the tube. An example of this manner of filling and sealing packages can be seen at http://www.packworld.com/machinery/bagging-amp-formfillseal/vertical-formfillseal-machine-liquids.

In some embodiments, a heat press method is applied to convert, for example, an article according to FIGS. 3a and 3b into an article of FIGS. 7a to 7c, by inserting an article of FIGS. 3a and 3b into a heat press having a heat sealing die of the desired seal pattern, and heat pressing to form the horizontal seals 406 and vertical seals 407 seen in FIG. 7b. Suitable time, temperature and pressure conditions for the heat press method can be adjusted for the particular materials.

In typical embodiments, first film layer 101 comprises a film formed from a transparent or translucent polymeric material. Suitable materials include polyolefins, such as low density polyethylene and particularly metallocene polyethylenes (e.g., those polyethylenes commercially available from Dow Chemical, Midland, Mich., under the trade designation “ENGAGE”) polyurethanes such as polyester or polyether polyurethanes (e.g., that polyurethane commercially available from B. F. Goodrich, Cleveland, Ohio, under the trade designation “ESTANE THERMOPLASTIC POLYURETHANE”), polyesters such as polyether polyester (e.g., that polyester commercially available from Du Pont Co., Wilmington, Del., under the trade designation “HYTREL POLYESTER ELASTOMER”), and polyamides such as polyether polyamides (e.g., those polyamides commercially available from ELF Atochem, North America, Inc., Philadelphia, Pa., under the trade designation “PEBAX RESINS”).

Furthermore, the film for first film layer 101 is flexible, and preferably somewhat elastomeric, to improve conformability when applied to a patient. For these reasons, the preferred films are polyurethanes, polyether polyesters, and polyether polyamides. First film layer 101 will typically have a thickness of less than about 200 microns, preferably between about 6 microns to about 130 microns, and most preferably between about 13 microns and about 52 microns.

At least a major portion of the exterior surface 103 of first film layer 101 is coated with a layer of adhesive (not shown). In some embodiments, the adhesive is a pressure sensitive adhesive. Although the entire area of exterior surface 103 may be coated with the adhesive, any major portion may be coated that allows the filled incise drape to serve its useful function, e.g., the adhesive need not coat the entire width or length of the drape. For example, non-coated portions may be included at any of the edges of the first film layer 101 to assist in removal of the drape from the patient or to assist in attachment of a handle to the film.

The adhesive layer coating the first film layer 101 is preferably a tacky pressure sensitive adhesive at body temperature that will adhere aggressively to the skin. Uniform attachment to the skin surface helps maintain a sterile surgical field. Aggressive adhesives are preferred due to the stress the first film layer 101 is under during surgery as a result of the retraction of the wound, the warm moist environment, and the abrasion the first film layer 101 may encounter as the surgeon's hands and instruments move in and out of the wound.

In some embodiments the adhesive layer coating first film layer 101 can releasably adhere to skin or to a surgical drape, permitting repositioning of the filled incise drape after an initial incision. For example, it can be desirable during surgery to remove the incision drape from a surgical drape after an initial incision, and position the incision drape temporarily in another location, e.g., a sterile location that could include a location on the surgical drape. In this way, access to an incision site during surgery can optionally be either through the incise drape or not through the incise drape. Subsequently, the incise drape can be repositioned over the incision site, for example, to introduce additional therapeutic fluid(s). Strength of the adhesive can be selected to permit removable adhesion of the incise drape to the surface of a surgical drape, without causing the surgical drape to peel away from a patient's body on removal of the incise drape from the surgical drape. Incise drapes 900 (see FIG. 14) and 950 (see FIG. 15) having adhesives that permit repositionable adhesion to a surgical drape can be particularly useful as repositionable depots of therapeutic material.

Suitable adhesives include acrylic adhesives, adhesives based on “KRATON” or “KRATON” polymers (“KRATON” and “KRATON” polymers are available from Shell Chemical Company, Houston, Tex.), rubber based adhesives such as those based on natural rubber, polyisobutylene, butylene rubbers and the like, polyurethane type adhesives, and polyvinylethyl ether and copolymers or blends thereof. Preferably, the adhesive also contains an antimicrobial such as iodine, triiodide complexes, lactam-triiodide complexes such as povidoneiodine, chlorhexidine salts such as chlorhexidine gluconate and chlorhexidine acetate, polymeric biguanides, polycationic antimicrobials such as polyhexamethylene biguanide, polyquaternium 1, octenidine alexidine, hexachlorophene, parachlorometaxylenol (PCMX), triclosan, phenols, fatty acid monoesters such as Lauricidin (glycerol monolaurate), quaternary surfactants, silver, and silver salts such as silver chloride, silver oxide and silver, hydrogen peroxide and the like.

The adhesive is preferably one of those described in U.S. Pat. Nos. 4,323,557; 4,931,282; 4,701,509; 4,732,808; 5,156,911; 5,017,625; and 5,204,110. Further, the adhesive 14 may be a continuous coating or may be a pattern coated as described in U.S. Pat. Nos. 4,798,201 and 5,290,615. These adhesive types may also include various chemical modifiers, e.g., tackifiers, crosslinkers, stabilizers, initiators, etc. to improve physical properties such as stability, viscosity, adhesion and the like.

In some embodiments, the pressure sensitive adhesive is covered by a release liner (not shown). The release liner includes an upper surface in contact with the pressure sensitive adhesive, and a lower surface. The upper surface and a lower surface extend between a leading edge and a trailing edge of the release liner. The leading edge of the release liner generally corresponds with a leading edge of first film layer 101 and the trailing edge of the release liner generally corresponds to a trailing edge of first film layer 101. Although all of the edges need not overlap (e.g., the release liner may be smaller or larger than first film layer 101), the release liner should fully cover the adhesive layer.

The release liner could be made of a variety of materials such as paper, plastic coated paper, plastic film, woven, non-woven, or knit textiles, as well as film textile laminates. The release liner may be hydrophilic to allow liquid absorbency or may be hydrophobic without absorbency. Preferred release liner materials include clear polymeric liners that allow the clinician to see through to the patient and thus accurately place filled incise dressing 100 during application of the filled incise dressing 100 to a patient as described further below. Preferred clear polymeric liners include polyolefins such as polyethylene and polypropylene, or polyester liners, as well as laminates such as polyolefin coated polyester. For products intended for gamma sterilization, use of a paper, polyethylene, polyester, or polyethylene coated polyester liner is preferred.

The release liner may be “cracked” (i.e., pre-cut) to permit peeling the release liner away from the adhesive layer on the filled incise drape.

One method manufacturing the filled incise drape involves coating an adhesive solvent solution onto the release liner, removing the solvent in an oven, and subsequently laminating this adhesive-coated release liner to the first film layer 101. Since the solvent is removed typically at elevated temperature in an oven, certain low melting thermoplastic polymeric release liners such as those made of low or medium density polyethylene may be adversely affected. And release liners incorporating a higher melting thermoplastic polymer such as a polyester layer, which are able to withstand the elevated temperature during drying, are not very flexible and can be quite noisy during application. A preferred approach is to form film liners by laminating polymers with high melting points and polymers with low melting points.

Desirable high melting point polymers for the preferred laminated film are characterized by having a melt temperature in excess of about 175° C. and preferably in excess of about 190° C. Polymers useful for this layer include but are not limited to polyester (e.g. polyethylene terephthalate; polybutylene terephthalate), polyamides (e.g. nylon 6,6; nylon 6), or cellulose acetate. The high melting point polymer layer should generally be present in the laminate in a total thickness (i.e., the sum total of all layers) of at least about 6 microns, preferably at least 12 microns and most preferably at least about 25 microns.

Desirable low melting point polymers for the preferred laminated film are characterized by having a melt temperature below about 175° C., preferably below about 150° C. Examples of polymers useful for this layer include polyolefins (e.g., polyethylene, polypropylene, polybutylene, ethylene/vinyl acetate, ethylene methylacrylate). The low melting point polymer layer should generally be present in the laminate in a total thickness (i.e., the sum total of all layers) of at least about 12 microns, preferably at least 25 microns and most preferably at least about 50 microns.

The preferred laminated film liner may be formed of two or more thermoplastic polymer layers, although one of the layers could be a thermoset if desired. For example, a high melting point polymer layer may be laminated on one or both sides by a low melting point polymer. In this manner, the high melting point polymer layer is able to support the stresses imparted in the drying oven while the low melting point polymer layer provides flexibility. In addition to the polymer layers, a low adhesion backsize (LAB) coating can be applied to one or both major surfaces of the multi-layered laminated film.

These laminated film liners may be formed by laminating premade films formed by any suitable method such as cast or blown extrusion. Alternatively, the laminates may be formed by coextrusion or extrusion lamination techniques.

A release coating of silicone, fluoro-chemical containing, long chain alkyl containing material, or other low surface energy coating, can be applied to the upper surface of the liner. This coating allows the liner to be peeled away from the adhesive with a force of less than about 120 g/cm, preferably less than 80 g/cm, more preferably less than 40 g/cm, and most preferably less than 25 g/cm when measured in a 180° peel at a speed of 225 cm per minute, at 25° C. and at 50 percent relative humidity. A preferred release coating is available from General Electric Company (Waterford, N.Y.), under the trade designation “GE SILICONE SS4331 LOW TEMPERATURE, FAST CURE PAPER PREMIUM RELEASE COATING”. The amount of the release coating will vary depending on the level of adhesion and coating thickness of the adhesive. A preferred polyethylene release liner is available from Rexam Release (Eagan, Minn.), under the trade designation “GRADE 10521 54 MIL NT LDP A16/000”. A preferred polypropylene liner is also available from Rexam Release under the trade designation “GRADE 15529D 2 MIL CL BOP EXP/000.

Embodiments of filled incise drapes of the present disclosure that include liquid in the at least one internal region (i.e., cavity) include liquid that is flowable. Uses of filled incise drapes can include delivery of flowable liquid compositions that can include any of: antimicrobials (i.e., antiseptics and antibiotics); lubricants for instrumentation; moisturizers to maintain tissue hydration and prevent tissue necrosis during surgery; hemostatic agents to prevent or reduce bleeding and blood loss; wound healing factors and anti-inflammatory agents; chemostatic factors to speed capillary ingrowth post-surgery; or compatible combinations of these.

Therapeutic material fillings of the present disclosure include a flowable liquid composition, which contains a medicament in a liquid vehicle. A large variety of medicaments are suitable for use in therapeutic material fillings of the present disclosure. The medicaments include, for example, an antimicrobial agent, an antiseptic, an antibiotic, an analgesic, a steroid, a growth factor, or chemotactic agents. A list of other possible medicaments that may be used include a thrombolytic agent, a fibrinolytic agent; a hemostatic agent; an antimicrobial agent or antibiotic; analgesics such as aspirin, methyl salicylate, camphor, menthol, a lower alcohol such as ethanol or isopropanol; local anesthetics such as lidocaine, benzocaine, priolocane, mixtures of these “EMLA” (i.e., Euctectic Mixture of Local Anesthetics, available from AstraZeneca), and the like; dexamethasone, dexamethasone sodium phosphate, dexamethasone acetate or another dexamethasone derivative, or another anti-inflammatory steroid or non-steroidal anti-inflammatory agent; a peptide, a protein, an enzyme (e.g., a proteolytic enzyme, collagenase, papain), a therapeutic quantity of a chaotropic agent (e.g., urea), and an antiviral agent.

Medicament compositions described herein can provide effective reduction, prevention, or elimination of microbes, particularly bacteria, yeast, and fungi, and in some cases viruses on the tissue to which it is applied, and thereby help to prevent or prolong the time to infection of the internal cavities (e.g., bladder, abdominal cavity, peritoneal cavity, trachea, lung, stomach, or upper sinuses). Since the contaminating microbes may be of a relatively wide variety, preferred compositions described herein have a broad spectrum of activity.

For example, the present disclosure provides methods of delaying the onset of an infection or preventing an infection caused by a microbial organism in an internal cavity of a subject upon contacting at least a portion of the interior surface of an opening into the internal cavity with an antimicrobial composition; and subsequently at least partially inserting an instrument into the opening. Other methods of the present invention include killing or inactivating microorganisms in at least a portion of the urethra of a subject by contacting at least a portion of the interior surface of the urethra with an antimicrobial composition; and subsequently at least partially inserting an instrument into the urethra.

Medicaments of the present disclosure include therapeutically-effective concentrations of one or more active agent that causes a local or systemic therapeutic biological response. The concentration of the medicament in the therapeutic material filling may be therapeutically effective before evaporation of the liquid vehicle, during evaporation of the liquid vehicle and/or after evaporation of the liquid vehicle.

The medicament should be compatible with the other components of the therapeutic material filling. A skilled person will recognize that the other components should not substantially interfere with the action of the medicament (e.g., by inactivation of the medicament; by sequestration of the medicament in a way that makes it substantially less accessible to the cells, tissue, organs or organisms).

A preferred medicament is an antimicrobial. The antimicrobial components can be antiseptics, antibiotics, or combinations thereof. In some embodiments, one or more antiseptics are used. Antiseptics are preferred due to the generally ability to kill bacteria faster and with broader spectrum with less or no chance of developing resistance. Although certain therapeutic material fillings of the present disclosure can have antimicrobial activity without any additional antimicrobial agents because of the incorporation of film-forming polymers that are inherently antimicrobial, additional antimicrobials can be added to the therapeutic material filling if desired. Preferably, the antimicrobial is present in the therapeutic material filling at a therapeutic concentration, in contrast to antimicrobials that are present in a composition as a preservative, as discussed below.

Herein, antiseptics are distinct from preservatives. Preservatives generally are used at very low levels since the purpose of these preservatives is to prevent bacterial growth in the therapeutic material filling, not to kill microbes on or in the tissue. They are typically added at levels less than 1% by weight and most often are at levels much less than 0.1% by weight. Typical preservatives include parabens, formaldehyde donors, 2-phenoxyethanol, benzyl alcohol; acids, such as benzoic acid, sorbic acid, citric acid, and salts thereof; quaternary ammonium surfactants such as benzalkonium chloride, and the like. When used on colonized or infected tissue at the industry standard preservative concentrations they would not achieve adequate antimicrobial activity.

A variety of antimicrobial agents may be included as long as they are compatible with the therapeutic material fillings (e.g., the components of the therapeutic material filling do not substantially prevent the activity of the antimicrobial agent, the antimicrobial agent does not substantially prevent the optical detection of the polymeric colorant). These antimicrobial agents include, but are not limited to, biguanides such as chlorhexidine salts such as chlorhexidine gluconate (CHG) and alexidine salts such as alexidine hydrochloride, and other cationic antiseptics disclosed in U.S. Published Patent Application No. 2006/0051385, which is incorporated herein by reference in its entirety; phenolic antiseptics such as parachlorometaxylenol (PCMX), triclosan, hexachlorophene, and others disclosed in U.S. Published Patent Application No. 2006/0052452, which is incorporated herein by reference in its entirety; fatty acid monoesters of glycerin and propylene glycol such as glycerol monolaurate, glycerol monocaprylate, glycerol monocaprate, propylene glycol monolaurate, propylene glycol monocaprylate, propylene glycol moncaprate, C8-C12 alkyl monoethers of glycerin and propylene glycol such as 2-ethylhexyl glycerin ether (available from Schuelke Mayr, Norderstedt, Germany, under the trade designation “SENSIVA SC 50”) as well as other antimicrobial lipids disclosed in U.S. Published Patent Application No. 2005/0058673, which is incorporated herein by reference in its entirety; hydrogen peroxide, natural oil antiseptics disclosed in U.S. Published Patent Application No. 2006/0051384, which is incorporated herein by reference in its entirety; surfactants and polymers that include a (C12-C22) hydrophobe and a quaternary ammonium group, polycationic amines such as polyhexamethylene biguanide (available from Arch Biocides under the trade designation “COSMOCIL CQ”) and polyquaternium 1 (available from Stepan, Northfield, Ill., under the trade designation “ONAMER M”), quaternary silanes, silver, silver salts such as silver chloride, silver oxide and silver sulfadiazine, octenidene, benzalkonium halides, cetyl pyridium halides, and the like, as well as combinations thereof. Various combinations of antimicrobial agents can be used in the therapeutic material fillings of the present disclosure. Suitable antiseptics include, for example: antimicrobial lipids; phenolic antiseptics; cationic antiseptics; iodine and/or iodophors; peroxide antiseptics; antimicrobial natural oils; or combinations thereof. These antiseptics are preferably present at a concentration greater than 0.1 weight percent (“wt %”) and preferably at least 0.25 wt % of the therapeutic material filling. Many therapeutic material fillings of this disclosure have the antiseptic present at greater than 0.5 wt % or even 1.0 wt % of the composition.

Examples of preferred antibiotics include neomycin sulfate, bacitracin, mupirocin, polymyxin, gentamycin, nitrofurantoin, sulfamethoxazole trymethoprim, rifampin, tetracycline, lysostaphin, and combinations thereof. Suitable antibiotic agents include, but are not limited to, beta-lactam antibacterials such as natural and synthetic penicillin type agents including penam penicillins (such as benzyl penicillin, phenoxymethyl penicillin, coxacillin, nafcillin, methicillin, oxacillin, amoxycillin, temocillin, ticarcillin, and the like), penicillinase-stable penicillins, acylamino and carboxypenicillins (such as piperacillin, azlocillin, mezlocillin, carbenicillin, temocillin, ticarcillin, and the like), and broader spectrum penicillins (such as streptomycin, neomycin, framycetin, gentamicin, apramycin, amikacin, spectinomycin, amoxycillin, ampicillin, and the like), cephalosporins, macrolides (such as tylosin, tilmicosin, aivlosin, erythromycin, azithromycin, spiramycin, josamycin, kitasamycin, and the like), lincosamides (such as lincomycin, clindamycin, pirlimycin, and the like), pleuromutilins (such as tiamulin, valnemulin, and the like), polypeptides, glycopeptides (such as vancomycin, and the like), polymyxins (such as polymyxin B, polymyxin E and the like), sulfonamides (such as sulfamethazine, sulfadiazine, silver sulfadiazine, sulfatroxazole, sulfamethoxypyridazine, sulfanilamide, sulfamethoxazole, sulfisoxazole, sulfamethizole, mafenide, and the like, alone or in combination with trimethoprim), chloramphenicol, thiamphenicol, florfenicol, tetracycline type agents (such as tetracycline, chlortetracycline, oxytetracycline, domeclocycline, doxycycline, minocycline, and the like), quinolones and fluoroquinolones (such as ciprofloxacin, enoxacin, grepafloxacin, levofloxacin, lomefloxacin, norfloxacin, ofloxacin, sparfloxacin, trovafloxacin, cinocacin, nalidixic acid, and the like), tiamulin, colistin, meropenem, sulbactam, tazobactam, methacycline, pyrimethamine, sulfacetamide, oxazolidinones, e.g., eperezoid, linezolid, N-((5S)-3-(3-fluoro-4-(4-(2-fluoroethyl)-3-oxy-1-p-iperazinyl)phenyl-2-oxy-5-oxazolidinyl)methyl)acetamide, (S)—N-((3-(5-(3-pyridyl)thiophen-2-yl)-2-oxy-5-oxazolidinyl)methyl)acetamide, 2,2-difluoro-N-({(5S)-3-[3-fluoro-4-(4-glycoloylpiperazin-1-yl)pheny-1]-2-oxo-1,3-oxazolidin-5-yl}methyl)ethanethioamide, (S)—N-((3-(5-(4-pyridyl)pyrid-2-yl)-2-oxy-5-oxazolidinyl)methyl)acetamide hydrochloride, and the like, aminoglycosides (kanamycin, tobramycin, netilmicin, and the like), aminocyclitols, amphenicol, ansamycin, carbaphenern, cephamycin, rifampicin, monobactam, oxacephem, streptogramins (such as quinupristin, dalfopristin, and the like), cycloserines, mupirocin, urea hydroxamates, folic acid analogs (such as trimethoprim, and the like), antibiotic-type antineoplastic agents (such as aclarubicin, actinomycin D, actinoplanone, aeroplysinin derivative, Nippon Soda anisomycins, anthracycline, azino-micyin-A, busucaberin, bleomycin sulfate, bryostatin-1, calichemycin, chromoximycin, dactinomycin, daunorubicin, ditrisarubicin B, doxorubicin, doxorubicin-fibrinogen, elsamicin-A, epirubicin, erbstatin, esorubicin, esperamicin-A1b, fostriecin, glidobactin, gregatin-A, grincamycin, herbimycin, idarubicin, illudins, kazusamycin, kesarirhodins, menogaril, mitomycin, mitoxantorone, mutamycin, mycophenolate mofetil, neoenactin, oxalysine, oxaunomycin, peplomycin, pilatin, pirarubicin, porothramycin, pyrindamycin A, rapamycin, rhizoxin, rodorubicin, sibanomicin, siwenmycin, sorangicin-A, sparsomycin, steffimycin B, talisomycin, terpentecin, thrazine, tricrozarin A, zorubicin, systemic antibacterials (such as 2,4-diaminopyrimidine), nitrofuran sulfones, narbofloxacin, and the like, and combinations thereof. If an antibiotic is used in therapeutic material fillings of the present disclosure, it may be used in combination with an antiseptic. The purpose of the antibiotic like the antiseptics is to kill microorganisms on or in mammalian tissue. They are not present as a preservative. Thus, therapeutic material fillings comprising antibiotics will have them present at greater than 0.1, 0.25, 0.5 and even 1.0% by weight. The concentration will depend on the composition, the antibiotic and the microorganism to be killed. Antiseptics are preferred over antibiotics since they are much less prone to resistance formation, faster acting, and often have a broader spectrum of antimicrobial activity than antibiotics.

For therapeutic material fillings wherein the medicament includes an antimicrobial agent, a particularly important property of the therapeutic material fillings is the ability to reduce the bacterial load on tissue, particularly skin, i.e., to kill the natural skin flora, rapidly. Preferably, antiseptic therapeutic material fillings of the present disclosure are capable of reducing normal skin flora by at least about 1 log₁₀ (i.e., 10-fold), more preferably by at least about 1.5 log₁₀, and most preferably by at least about 2 log₁₀ (i.e., 100-fold), in 2 minutes on a dry human skin site (typically, skin on an abdomen or back) using ASTM testing method E1173-93 and a 30-second scrub with gauze soaked in the therapeutic material filling using moderate pressure.

Therapeutic material fillings of the present disclosure optionally may comprise other optional ingredients that may be delivered to the skin using a composition of the present disclosure and include components of cosmetic compositions. These include, but are not limited to, emollients, humectants, conditioners, moisturizers, vitamins, herbal extracts, antioxidants, exfoliants such as α-hydroxy acids or β-hydroxy acids, emulsifiers, skin soothing agents, and skin sensates, hemostatic agents such as chitosan, thrombin, fibrinogen, protamine, epinephrine, desmopressin, vitamin K, and the like; local anesthetics such as benzocain, prilocaine, lidocaine and the like, and combinations of any two or more of the foregoing ingredients.

In some embodiments, the flowable liquid composition may contain a polymeric component. Alternatively, one or more polymers may be added to the flowable liquid composition. The polymer may be soluble or dispersible in the composition. Preferred dispersions are physically stable and do not require shaking prior to use. In some embodiments suitable polymers include, for example, polymers derived from vinylic polymers and in particular acrylate monomers such as those described in U.S. Pat. Nos. 6,838,078; 4,584,192; 4,542,012; 7,459,167; and 6,605,666; each of which is incorporated herein by reference in its entirety. In certain preferred embodiments, the polymeric component of the composition is substantive and resists removal by any constituent of blood or other bodily fluid (e.g., urine, semen, saliva, mucous, tears, sweat, cerebrospinal fluid,) as well as saline and other fluids used in surgical irrigation.

In some embodiments, the polymeric component is water-soluble or water-dispersible. Advantageously, water-soluble or water-dispersible polymers can exist in a relatively stable, uniform distribution in an aqueous liquid vehicle. In some embodiments, the polymeric component s water-insoluble. In these embodiments, the polymeric component may be used as a dispersion in an aqueous system or it may be used as a solution or as a dispersion in a hydroalcoholic solution. Notably, the polymeric components may serve as polymeric emulsifiers which help emulsion stability. The polymeric emulsifier may be used to help stabilize water in oil (w/o), oil in water (o/w) or multiple emulsions.

Therapeutic material fillings of the present disclosure comprise a liquid vehicle, in which the medicament and optional polymeric component are dispersed and/or dissolved. Suitable liquid vehicles include water, optionally in combination with acetone or an alcohol, particularly a (C1-C4) alcohol (i.e., a lower alcohol) such as ethanol, 2-propanol, and n-propanol; glycols such as glycerin, propylene glycol, dipropylene glycol, polyethylene glycol and mixtures thereof. The preferred liquid vehicle is injectable-grade water, i.e., USP grade “water for injection”; however, other forms of purified water may be suitable such as distilled and deionized water. Aqueous solutions free of volatile flammable solvents are preferred.

It may be desirable for the liquid vehicle to include a lower alcohol such as ethanol, isopropanol, or n-propanol. These alcohols are well known to have antiseptic activity and to contribute to rapid microbial kill. For these applications the alcohol to water ratio is preferably at least about 60:40, and more preferably at least about 70:30, by weight. Addition of alcohol in these high concentrations can also decrease the dry time of the composition on the skin, when desired. Such materials must be used with caution to avoid ignition from sources such as an electrocautery.

When a lower alcohol is used, incorporation of surfactants may or may not be necessary. In some embodiments, the addition of a surfactant can improve antimicrobial efficacy, as described in U.S. Pat. No. 7,147,873, which is incorporated herein by reference in its entirety. Notably, anionic and zwitterionic surfactants can be particularly effective at enhancing the antimicrobial efficacy. Inclusion of ionic or nonionic surfactants can be used to increase compatibility of medicament or polymeric component with the vehicle solution. Examples of nonionic surfactants useful in this role may include polyethoxylates, nonyl phenyl polyethoxylates, alkyl esters, alkyl polyethoxylates, and PEG-PPG copolymers. Examples of ionic surfactants include alkyl or aryl quaternary ammonium compounds, alkyl or aryl amines, fatty acid salts, aryl or alkyl sulfonates.

Certain preferred therapeutic material fillings include water and are substantially free (i.e., less than about 10 wt-%; more preferably, less than about 5%; even more preferably, less than about 3%) of volatile organic solvents (i.e., those having a closed-cap flash point of greater than about 140° F. (60° C.)), such as acetone, lower alcohols, alkanes, volatile silicones. The addition of lower alcohols (C1-C4) at less than about 4 wt-% may improve wetting of the therapeutic material fillings and yet maintain a flashpoint above about 140° F. (60° C.). Flashpoint is measured according to test method ASTM D3278-96.

Aqueous formulations of therapeutic material fillings are preferred since these formulations are gentle to both skin and mucosal tissue and may even be suitable for use on open wounds as a wound cleanser.

In some embodiments, the at least one interior region of the incise drape is filled with a lubricant. Lubricants are generally aqueous, glycol (glycerin, propylene glycol, PEG etc), or hydrocarbon such as petrolatum.

Kits are also contemplated. In any embodiment, a kit may further optionally include one or more of the following components: a percutaneously implantable device, one or more pre-packaged sterile liquid therapeutic material(s), a rinse composition, an incise drape, an incision wound edge protection device, a lubricant; and a scalpel or other surgical instrument. An example of an incision wound edge protection device is available from Applied Medical Resources, Rancho Santa Margarita, Calif., under the trade designation “ALEXIS O”. In any embodiment, a kit may include instructions for preparing a skin site for a surgical procedure. In any embodiment, a kit may include instructions for using the filled surgical drape. Kits of the present disclosure may comprise a liquid-filled drape of the present disclosure in combination with any of: an adhesive coated film incision drape (i.e., an incise drape, such as any of those available from 3M Company, St. Paul, Minn., under the trade designations “IOBAN 2”, “STERIDRAPE”, “STERIDRAPE 2”, or from Smith and Nephew, under the trade designation “OPSITE”). In these embodiments, the composition comprises a medicament, as described above, in a suitable carrier vehicle as described above. In any embodiment of the kit, the medicament may comprise an antimicrobial agent wherein, when the therapeutic material filling is contacted for a period of time with an area of a surface (e.g., a skin surface), having a number of cultivable microorganisms present thereon, the concentration of antimicrobial agent in the liquid vehicle is sufficient to reduce the number of cultivable microorganisms in the area. In any embodiment, the kit further can comprise a plurality of sealed containers, each of the plurality of containers containing the therapeutic material filling. In some embodiments, the plurality of sealed containers may comprise a first container containing a first therapeutic material filling and a second container containing a second therapeutic material filling. The first and second therapeutic material fillings may comprise the same medicament or a different medicament.

In another embodiment of a kit of the present disclosure, the incise drape may be included without being filled with the liquid, and the liquid may be injected before or after application to the patient. For example, the liquid could be injected into the drape through a septum or through any elastomeric film or port.

The configuration of the filled incise drape and medicament can be selected according to the method (e.g., preparation of a skin site for surgery or for the treatment of a medical condition) in which the contents of the kit will be used. In any embodiment of the kit, the medicament may comprise an antimicrobial agent wherein, when the therapeutic material filling is contacted for a period of time with an area of a surface (e.g., a skin surface), having a number of cultivable microorganisms present thereon, the concentration of antimicrobial agent in the liquid vehicle is sufficient to reduce the number of cultivable microorganisms in the area. In any embodiment, the kit further can comprise a plurality of sealed containers, each of the plurality of containers containing the therapeutic material filling. In some embodiments, the plurality of sealed containers may comprise a first container containing a first therapeutic material filling and a second container containing a second therapeutic material filling. The first and second therapeutic material fillings may comprise the same medicament or a different medicament.

In any embodiment, the kit may further comprise a rinse or remover composition for removing the composition from a treated surface (e.g., a treated skin surface). In some embodiments, the rinse solution can contain water (e.g., deionized water or water for injection). The rinse solution may further comprise a component (e.g., soap; an organic solvent acceptable for use on skin, such as ethanol, isopropanol, and/or acetone, for example) to assist the cleaning and removal of the composition. In any embodiment, the rinse solution may further comprise an agent that counteracts an effect of the medicament. That is, in addition to reducing the effect of the medicament by dilution, the rinse may comprise an agent (e.g., a chemical agent) that directly or indirectly inhibits the effect of the medicament.

Therapeutic material fillings of the present disclosure can be used in a variety of methods. In some embodiments, the therapeutic material fillings can be used in a method of any of making an incision, a percutaneous injection, a percutaneous insertion of a medical device, or a surgical procedure. In these embodiments, the composition may comprise a medicament comprising, for example, an antimicrobial agent, an analgesic agent, an anesthetic agent (e.g., a local anesthetic) or a combination of any two or more of the foregoing medicaments.

Preferably, applying therapeutic material fillings of the present disclosure results in reducing normal skin flora by at least about 1 log₁₀ (i.e., 10-fold), more preferably by at least about 1.5 log₁₀, and most preferably by at least about 2 log₁₀ (i.e., 100-fold), in 2 minutes on a dry human skin site (typically, skin on an abdomen or back) using ASTM testing method E1173-93 and a 30-second scrub with gauze soaked in the composition using moderate pressure.

In any embodiment, the methods further may comprise performing a percutaneous insertion of a medical device. In any embodiment of the methods, applying the composition to the skin site may comprise applying the composition in the form of a liquid, aerosol or foam. Suitable foaming agents include, for example, silicone copolyols and fluorinated surfactants. Applicators for applying liquid compositions to skin are known in the art and these applicators may be used to apply compositions of the present disclosure. Nonlimiting examples of suitable applicators include the applicators described in U.S. Pat. Nos. 5,435,660; 6,248,085; 7,261,701; 7,377,710; 6,672,784; and 6,422,778; each of which is incorporated herein by reference in its entirety.

### Embodiments

Item 1. An article, comprising:
   an enclosure comprising:
   a first film layer;
   a second film layer, wherein the first film layer and the second film layer are sealed together to form at least one interior region for enclosing a liquid; and
   an adhesive layer disposed on an exterior surface of the first film layer; wherein the at least one interior region is liquid-filled.
Item 2. The article of item 1, wherein the liquid in the at least one interior region comprises a therapeutic material.
Item 3. The article of item 1, wherein the first film layer and second film layer are sealed together to form a plurality of interior regions for enclosing liquid, each interior region having a sealed outer periphery.
Item 4. The article of item 3, wherein each interior region in the plurality of interior regions is liquid filled.
Item 5. The article of item 3, wherein at least a first interior region is filled with a first liquid and at least a second interior region is filled with a second liquid.
Item 6. The article of item 5, wherein at least the first liquid is a therapeutic material.
Item 7. The article of any preceding item, wherein the adhesive layer is a continuous layer on the exterior surface of the first film layer.
Item 8. The article of any preceding item, further comprising a release liner layer releasably adhered to the adhesive layer.
Item 9. The article of any preceding item, further comprising a port in fluid communication with interior region of the at least one interior region.
Item 10. The article of any preceding item, further comprising a plurality of fluid channels defined within the at least one interior region.
Item 11. The article of any preceding item, wherein the entire article is sterile and the article is packaged in a hermetically sealed outer package.
Item 12. A method, comprising:
   providing an enclosure comprising:
      a first film layer;
      a second film layer;
      at least one interior region; and
      a composite layer comprising the first film layer and an adhesive layer disposed on an exterior surface of the first film layer;
      wherein:
         the composite layer is liquid-impervious and flexible;
         the first film layer and the second film layer are sealed together to form the at least one interior region;
         the interior region has a sealed outer periphery; and
         the interior region is liquid-filled;
   positioning the article over an intended site of operation, with the adhesive layer facing towards the intended site of operation;
   penetrating each of the first film layer, the at least one interior region, and the composite layer with a surgical device; and
   penetrating the intended site of operation with the surgical device.
Item 13. The method of item 12, wherein positioning the article over the intended site of operation comprises contacting the adhesive layer of the article to a skin surface surrounding the intended site of operation.
Item 14. The method of item 12, wherein positioning the article over the intended site of operation comprises contacting the adhesive layer of the article to a major surface of a surgical drape positioned over at least a portion of the intended incision site.
Item 15. A kit, comprising:
   an article according to any one of items 1 to 11;
      optionally, a percutaneously implantable device;
      optionally, one or more pre-packaged sterile liquid therapeutic material(s);
      optionally, a rinse composition;
      optionally, an incise drape;
      optionally, an incision wound edge protection device;
      optionally, a lubricant; and
      optionally, a medical instrument.

1. An article, comprising:
a liquid impervious enclosure comprising:
a first film layer;
a second film layer, wherein the first film layer and the second film layer are hermetically sealed together to form at least one interior region enclosing an antimicrobial liquid composition; and
a pressure sensitive adhesive layer disposed on an exterior surface of the first film layer.

2. The article of claim 1, wherein the antimicrobial liquid composition in the at least one interior region comprises a therapeutic material.

3. The article of claim 1, wherein the first film layer and second film layer are sealed together to form a plurality of interior regions for enclosing liquid, each interior region having a sealed outer periphery.

4. The article of claim 3, wherein each interior region in the plurality of interior regions is liquid filled.

5. The article of claim 3, wherein at least a first interior region is filled with the antimicrobial liquid composition and at least a second interior region is filled with a second liquid.

6. The article of claim 5, wherein at least the second liquid comprises a therapeutic material.

7. The article of claim 1, wherein the pressure sensitive adhesive layer is a continuous layer on the exterior surface of the first film layer.

8. The article of claim 1, further comprising a release liner layer releasably adhered to the pressure sensitive adhesive layer.

9. The article of claim 1, further comprising a port in fluid communication with the at least one interior region.

10. The article of claim 1, further comprising a plurality of fluid channels defined within the at least one interior region.

11. The article of claim 1, wherein the entire article is sterile and the article is packaged in a hermetically sealed outer package.

12. A method, comprising:
providing the article of claim 1;
positioning the article over an intended site of operation, with the pressure sensitive adhesive layer facing towards the intended site of operation;
penetrating each of the first film layer, the at least one interior region, and the second film layer with a surgical device; and
penetrating the intended site of operation with the surgical device.

13. The method of claim 12, wherein positioning the article over the intended site of operation comprises contacting the pressure sensitive adhesive layer of the article to a skin surface surrounding the intended site of operation.

14. The method of claim 12, wherein positioning the article over the intended site of operation comprises contacting the pressure sensitive adhesive layer of the article to a major surface of a surgical drape positioned over at least a portion of the intended incision site.

15. A kit, comprising:
the article according to claim 1; and at least one of
a percutaneously implantable device;
one or more pre-packaged sterile liquid therapeutic material(s);
a rinse composition;
an incise drape;
an incision wound edge protection device;
a lubricant; and
a medical instrument.
